(19) United States
(12) Patent Application Publication
(10) Pub. No.: US 2013/0189745 A1
(43) Pub. Date: Jul. 25, 2013

(54) ARTIFICIAL CELLULOSOME AND THE USE OF THE SAME FOR ENZYMATIC BREAKDOWN OF RESILIENT SUBSTRATES

(71) Applicants: Wolfgang H. Schwarz, München (DE); Jan Krauss, Freising (DE); Vladimir V. Zverlov, München (DE); Daniel Hornburg, Freising (DE); Daniela Köck, Freising (DE); Louis-Philipp Schulte, München (DE)

(72) Inventors: Wolfgang H. Schwarz, München (DE); Jan Krauss, Freising (DE); Vladimir V. Zverlov, München (DE); Daniel Hornburg, Freising (DE); Daniela Köck, Freising (DE); Louis-Philipp Schulte, München (DE)

(73) Assignee: Technische Universitat Munchen, Munchen, DE (US)

(21) Appl. No.: 13/810,920

(22) PCT Filed: Jul. 19, 2011
(86) PCT No.: PCT/EP2011/003617

§ 371 Date: Feb. 5, 2013

(30) Foreign Application Priority Data
Jul. 20, 2010 (EP) 10007525.8

(51) Int. Cl. C12N 9/96

(52) U.S. Cl. CPC C12N 9/96
USPC 435/99; 435/188

(57) ABSTRACT

The present invention relates to an in vitro produced, artificial cellulosome for enzymatic breakdown of resilient substrates. In particular, the present invention provides a complex having an increased activity on resilient substrates, such as crystalline cellulose. The in vitro formed complex comprises a backbone scaffold having at least four binding sites capable of binding the enzyme components, whereby at least two of the binding sites have essentially the same binding specificity; and at least three different enzyme components being randomly bound to the at least four binding sites. Method for preparing the complex and uses of the same for enzymatic breakdown of resilient substrates are also provided.

### BACKGROUND OF THE INVENTION

1. Field of the Invention

The present invention provides an artificial cellulosome for enzymatic breakdown of resilient substrates. In particular, the present invention provides a complex comprising a backbone scaffold having at least four binding sites capable of binding the enzyme components, whereby at least two of the binding sites have essentially the same binding specificity; and at least three different enzyme components being randomly bound to the at least four binding sites. In addition, the present invention relates to a method for preparing the complex. Further, the present invention relates to the use of said complexes as well as the different enzyme components for enzymatic breakdown of resilient substrates, such as cellulose.

2. Description of the Related Art

Cellulose is an abundant renewable source for biotechnology to produce biofuels and building blocks for the chemical industry. Cellulose from lignocellulosic biomass is set to become the largest source of sugar for industrial scale fermentation with the arrival of the upcoming second generation of White Biotechnology. Glucose for industrial fermentation is currently produced primarily from starch. The utilization of cellulose for sugar production would at least double the per hectare yield of agricultural products. In addition, the acreage of arable land could be increased because a greater variety of energy plants would be planted, including those which grow in mediocre soil, under unfavorable climatic conditions, as well as in dry, wet, cold or salt-rich environments.

Cellulose however is a recalcitrant material, refractory both to enzymatic as well as to chemico-physical degradation. Cellulose consists of long fibers of linear molecules without branches. It is chemically a highly homogenous β-1,4-glucan which forms regular crystals of the form Iα and Iβ¹. However, the crystals are not perfectly structured they are more or less regularly interrupted by amorphous regions. The structural features of cellulose are therefore numerous and require enzymes of various modes of breakdown.

Present day technology for enzymatic hydrolysis of cellulose has to use large quantities of enzymes usually of fungal origin for the degradation of cellulosic material. These enzymes usually cannot be recycled. Hydrolysis is rather inefficient due to a number of reasons: heterogeneity of the material, complexation with hemicellulose, pectin and lignin, crystallinity of the cellulose, lack of accessibility for enzymes due to tight packaging in cell walls and crystals etc. This increases the number of different enzyme activities needed for degradation as well as reduces the reaction velocity and therefore the yield of the process. By raising the reaction temperature, the reaction velocity can be increased, but at a cost to enzyme stability. This is especially intricate for the enzyme group of the cellulases which are intrinsically slowly reacting enzymes that have to cope with a crystalline, i.e. insoluble substrate. The diversity of cellulase actions on cellulose explain the multiplicity of the different enzymes involved in cellulase complexes: the structural heterogeneity of the cellulose fibre (crystalline or amorphous, edges or planes etc.), the type of crystal (Iα or Iβ), the mode of activity (processive exo-glucanase or cellobiohydrolase, processive or non-processive endo-glucanase, β-glucosidase). These enzymes have to function in harmony for efficient degradation of the crystalline substrate, although they all cleave an identical β-1,4-glucosidic bond.

Commercially available cellulases generally used in the White Biotechnology contain a mixture of soluble enzymes of fungal origin. The most successful producers are among others Trichoderma longibrachiatum, T. reesei (=Hypocrea jecorina), T. viride (=T. harzianum or Hypocrea atroviridis), Aspergillus niger, Phanerochaete chrysosporium, Chrysosporium lucknowense and Penicillium janthinellum. Some mixtures of cellulases are prepared from two microorganisms such as from T. longibrachiatum and A. niger, or from T. longibrachiatum and T. reesei. These fungi produce high amounts of exoproteins in their culture fluid, partially after intensive strain development by selection and by genetic engineering. The cellulases comprise endo-glucanases, cellobiohydrolases (exo-glucanases) and β-glucosidases, in some strains also a number of hemicellulases.

For this new technology a dramatic increase in demand for enzyme formulations is predicted for the hydrolysis of sustainably produced renewable sugar sources, such as lignocellulosic material. Other sectors of cellulase use are mainly food, textiles, detergents, paper industry and additives to animal feed. Although cellulases already have a big market, future fields of use are generally expected to be dominated by the production of cellulases for the emerging and potentially much bigger market of biomass degradation in the biofuel and bulk chemical biotechnology sector.

However, the different structural characteristics of cellulose crystals and their insoluble nature require the simultaneous presence of several different activities such as processive and non-processive cellulases. The single activities have a lower activity if present alone; only in combination the enzymes show high activity. This difference between single and multiple enzymes in a mixture is called synergism, where the activity of the mixture is higher than the sum of the single activities. However, this synergy is exerted by the fungal enzymes in a soluble mixture of single enzymes which are not “complexed”, i.e. packed together by adsorption or in a polypeptide Synergy between soluble, single enzymes precludes the presence of at least two co-working activities at one site of the substrate. In soluble systems this is only possible with a high concentration of enzymes in the mixture. Examples of the limitations of fungal cellulases are the relatively high concentration necessary for enzymatic hydrolysis, the limited thermostability, and the high abortive binding rate. Such limitations have to be overcome for higher performance.

The commercially available cellulases are dominated by fungal enzymes. However, bacterial enzyme systems have also been investigated intensively. The soluble enzymes of Thermomonospora bispora or of other thermophilic aerobic bacteria have been discussed as additives for fungal cellulase mixes. Some anaerobic bacteria have been described whose extracellular enzyme systems have a higher specific activity and processivity on cellulose. Most of the latter produce a large extracellular enzyme complex which binds the single enzymes on a backbone scaffold, the so called scaffoldin or Cip (cellulosome integrating protein). These complexes are held together by strong protein-protein interactions which are species specific. These complexes are called cellulosomes. Relatively few bacteria are known to produce cellulosomes. Their list comprises so far:

### In the Phylum Firmicutes:

Lachnospiraceae—Butyrivibrio fibrisolvens, Ruminococcus flavefaciens, R. albus;
Clostridiaceae—Clostridium cellulovorans, C. cellobioparum, C. papyrosolvens, C. josui, C. cellulolyticum, C. thermocellum, C. sp. C7, Bacteroides sp. P-1, B. cellulosovens, Acetivibrio cellulolyticus;

### In the Phylum Actinobacteria:

Nocardiopsaceae—Thermobifida (Thermonospora) fusca;

### In the Phylum Fibrobacteres:

Fibrobacteriaceae—Fibrobacter succinogenes.

Some improvement in the analysis of the efficiency of the cellulosome could be achieved with the strictly anaerobic, thermophilic bacterium Clostridium thermocellum which is the microorganism with the fastest growth rate on the recalcitrant substrate crystalline cellulose as it has one of the most efficient enzymatic cellulose degradation systems³. Without being bound to theory, some evidence is accumulating that this higher efficiency over other cellulolytic systems is due to the formation of a huge enzyme complex which however cannot be produced in industrial amounts. The complex has a diameter of ˜18 nm and a mass in excess of 2×10⁶ Da⁴. About 30 dockerin containing, cellulosome related genes have been more or less accidentally cloned by screening genomic libraries from C. thermocellum for enzymatically active clones^{5,6}. In addition, the scaffoldin protein CipA, which contains 9 type I cohesin modules to which enzymes and other protein components specifically dock by virtue of their type I dockerin modules⁷ was identified. Type II cohesin-dockerin interactions anchor the CipA protein to the cell wall bound proteins OlpB or SdbA and possibly to others^{8,9}. The non-enzymatic component CspP is presumably involved in structure formation of the huge complex¹⁰. However, not much is known about the structure of the complex and how it is assembled. Cellulosomes investigated from other bacteria also contain a scaffoldin protein, often with a different architecture.

72 cellulosomal genes were identified in the genomic sequence¹¹ of Clostridium thermocellum ATCC 27405¹². The most prevalent cellulosomal components were identified by proteome analysis of isolated cellulosomes and by mRNA analysis. However, it was by no means clear which of the components were indispensable to cellulose breakdown, and what role the complex formation could play.

Only the partial reconstitution of the cellulosome by construction of small tertiary complexes of a mini-scaffoldin combined with two recombinantly produced enzyme components was possible so far and showed a distinct synergistic effect¹⁴. The mutant isolated by another research group, called AD2, did not absorb to cellulose, but was not characterized in respect to its molecular mechanism, cellulose degrading ability and cellulosome formation 15,16. From mutagenized cultures of C. thermocellum non-cellulosome-forming mutants were isolated which did not adsorb to crystalline cellulose 17.

Other groups construct backbone scaffolds with e.g. three divergent cohesin modules for the targeted equimolar binding of the same number of different enzyme components (such as in WO=2010057064). A complex cellulosome structure is assembled on a yeast cell surface using a constructed “yeast consortium”. In contrast to the complex of the invention only non-statistical (ordered) binding is intended and the ratio of the components cannot be adjusted as this is possible with the present invention.

In WO=2010012805 a carbohydrate binding module and the X-module from the cellulosomal scaffoldin are used to enable better production and secretion of proteins in the recombinant host. In this case a cellulase gene is genetically fused to the polypeptide chain, where CBM and the X modules are used as “helper” modules for expression and not with the purpose to lead for optimized cellulose break-down.

In US20090035811 the cohesin containing proteins and the enzymatic cellulases are in vivo produced by yeast cells and stay attached to the cell surface. This leads to overload of enzyme complexes on the relatively large cell surface. It is not shown how the composition of the enzyme components and their ratio can be manipulated. The cellulosome producing organism (yeast) cannot easily be adapted to a composition suitable for another substrate.

In contrast to the non-cell bound system of the present invention, the yeast-cell bound cellulosomes as described in the art have the disadvantage of being bound to one specific product, depending on the organism in which it is engineered. The efficiency cannot be optimized by changing the ratios of components. Further, native cellulosomes, such as yeast-cell bound cellulosomes cannot be produced in industrial amounts.

All three methods do not lead to a cellulase activity higher than the natural cellulosome, probably due to suboptimal complex composition which cannot be accustomed to the needs of the substrate.

In vitro assembly of the cellulosome and its single components would nevertheless be necessary to investigate the role of the single genes in cellulolysis and fiber degradation. Such attempts have however failed so far due to the insurmountable difficulty in taking the components apart in their native state—the tight binding in the complex prevents easy separation with mild, non-denaturing methods.

Enzymatic breakdown of insoluble and crystalline material such as crystalline cellulose and heterogenous hemicellulose is still inefficient, slow and requires a high enzyme concentration, which makes industrial exploitation costly and relatively ineffective with present day enzyme preparations.

It is therefore an object of the present invention to provide new enzyme formulations capable of enzymatic breakdown of resilient substrates, such as crystalline cellulose and heterogenous hemicellulose with higher effectivity.

It is another object of the present invention to provide new enzyme formulations as well as effective and cost efficient in vitro methods and uses of these enzyme formulations which overcome the drawbacks of the prior art enzyme mixtures, especially that lower enzyme concentration is needed for the enzymatic breakdown, the thermostability of the enzyme formulation is enhanced, and the binding rate of the enzyme is improved.

### SUMMARY OF THE INVENTION

According to the present invention, a strong enhancement of activity could be achieved by the complex of the invention as explained in more detail below. The enhancement of the activity of such an enzyme system responsible either for a continuous chain of catalytic events or a synergistic action on resilient substrates, including but not limited to crystalline cellulose and heterogeneous hemicellulose, is demonstrated herein. Surprisingly, the inventors could show that the complexes of the invention, when reconstituted in vitro, exhibit higher activity on crystalline cellulose than the native cellulosomes isolated from the bacterium.

The present inventors isolated mutants of C. thermocellum which did not form complexes and instead secreted native cellulosomal components in a non-complexed form. These proteins were initially used to reconstitute an artificial cellulosome having enhanced activity. With this mutant the role of synergism could now be investigated for the first time by in vitro reconstitution of the complexes.

Thus, the invention provides a particle-bound or particle-free complex comprising: (a) a backbone scaffold comprising at least four binding sites, wherein at least two of the binding sites having essentially the same binding specificity; and (b) an enzyme component bound in vitro to each of said four binding sites, wherein at least three of said enzyme components are different enzyme components. The complex contains a molar ratio of 1:1 of the cohesin modules in the backbone scaffold (a) to the sum of dockerin containing enzyme components (b). The at least three enzyme components (b) are preferably present in the complex in a in a molar ratio to each other of 1:1 to 1:50, 1:1.5 to 1:30, preferably 1:1.8 to 1:15 of the backbone scaffold.

In a preferred embodiment, the complex is a particle-free or isolated complex; which is not bound to a living cell, particularly preferred not bound to a yeast cell.

Preferably, the said enzyme components are randomly bound in vitro to the at least four binding sites.

The term “in vitro” as used herein means separated from a living cell or organism.

The term “complex” or “enzyme complex” as used herein means a coordination or association of components linked by chemical or biological interaction. Said complexes may be linked together to form a higher order complex (also synonymously used herein with “artificial cellulosome” or “cellulase complex”) consisting of one or more cohesin containing backbone scaffolds, preferably cohesin containing scaffolding proteins (also designated herein with “mini-scaffoldin”) and one or more dockerin containing enzymatic or non-enzymatic components, as explained in more detail below. Alternatively, the artificial cellulosome consists of one or more dockerin containing backbone scaffolds, preferably dockerin containing scaffolding proteins (also designated herein with “mini-scaffoldin”) and one or more cohesin containing enzymatic or non-enzymatic components. In contrast, the term “enzyme mixture” as used herein relates to industrially produced soluble enzymes.

The term “particle-bound” complex as used herein means that the complex of the invention is bound to particles which serve as a carrier material. Suitable particles are for example nano-particles. Nano-particles used in this technology are smaller than 2000 nm, preferably with a mean diameter smaller than 100 nm. They may consist of organic or inorganic material such as silicon, metal oxide, gold, polystyrol and other organic polymers, and other non-living materials, or hybrids of different materials (such as core-shell nanoparticles). Preferably ferromagnetic nanoparticles are used which exhibit superparamagnetic behavior. More preferably their core is coated with a polymeric shell such as polystyrol, and the surface of the particles is chemically modified to allow chemical coupling of biomolecules. Preferably the modifications are free carboxyl groups (COOH) or free amino groups (NH₂) for coupling reactions with crosslinking agents to couple proteins or chemical backbone molecules.

Alternatively, the nanoparticle surface, preferably modified with amine or carboxy functional groups, can be covalently crosslinked preferably to a heterobifunctional molecule such as a polyethyleneglycol-based linker and finally to nitrilo-triacetic acid (NTA) by glutaraldehyde (amine modification) or EDC/Sulfo-NHS (carboxy modification) respectively as is state of the art. Miniscaffoldin backbone molecules are attached to the NTA residues via their poly-histidine fusions on the protein ends (preferably 6×His tagged) by using state of the art nickel affinity technology.

The nanoparticles covered with the backbone scaffolds are mixed with enzymes as described in the reconstitution of the complexes.

The term “particle-free” as used herein means the complex of the invention is non-cell bound or isolated, respectively.

The term “backbone scaffold” as used herein relates to a support used as a backbone for the complex which provides for suitable binding sites for enzymatic or non-enzymatic protein components. The backbone may be a backbone protein, a scaffolding backbone or a polymeric organic molecule with multiple binding sites. A scaffolding backbone may consist of one or more mini-scaffoldins.

The term “having essentially the same binding specificity” when used in reference to the binding sites for the enzyme components refers to the specificity of binding between cohesin and dockerin modules, whereby only cohesin-dockerin pairs of identical binding specificity bind each other. This can be tested e.g. by mixing a pair of proteins and estimating the running behaviour in native gel electrophoresis.

In one embodiment, the invention relates the complex as defined herein, wherein the backbone scaffold is linear. The linear backbone scaffold may be of synthetic or biologic origin. A synthetic backbone scaffold may be for instance a synthetic polymer carrier or a linear organic polymer with functional groups capable of binding proteins. The proteins can be the enzymes to be included in the complex, or proteins containing one or more modules for taking part in cohesin-dockerin interaction (which bind the enzyme components). A biologic backbone scaffold may be a protein having naturally occurring binding sites (cohesins) for dockerins fused naturally or by genetic engineering to the enzyme components or binding modules.

In a further embodiment of the invention, enzyme components are bound to the linear backbone scaffold by a cohesin-dockerin interaction. In a preferred embodiment of the invention, the backbone scaffold of the complex of the invention has at least four cohesin binding sites for dockerins.

Preferably the backbone of the complex of the invention consists of one or more proteins, wherein the one or more proteins are backbone proteins which are linked together by chemical interaction or by a cohesin-dockerin interaction which is different in binding specificity from that in the backbone enzyme interaction, whereby the binding specificity of the linking interaction is different from the binding specificity of the enzymes. More preferably, the one or more proteins are linked by a cohesin-dockerin interaction having a binding specificity which is different from the binding specificity of the cohesin-dockerin interaction binding the enzyme components.

The term “cohesin-dockerin interaction” as used herein refers to the interaction between a cohesin and a dockerin. Dockerin is a protein module found in the components of the cellulosome, preferably one in each enzyme component of the cellulosome. The dockerin's binding partner is the cohesin module which is a usually repeated modular part of the backbone scaffold protein in cellulosomes. This interaction is essential to the construction of the cellulosome complex. For binding the different enzyme components to the complex, the same cohesin-dockerin system is used in the complex of the invention. One or more of the backbone scaffold proteins of the invention may be linked by cohesin-dockerin interaction; whereby this cohesin-dockerin pair has a different binding specificity than the cohesin-dockerin pair used for binding the enzyme components. The binding of the components to a polysaccharide can among other methods be determined by retardation of protein bands in native gel electrophoresis, or by measuring the amount of proteins after separating the liquid fraction and the solid fraction containing the cellulose particles with a standard technology for protein concentration determination as is known by a person skilled in the art.

In a further embodiment, the backbone scaffold of the complex of the invention is derived from a non-catalytic scaffolding protein from cellulolytic, cellulosome forming microorganisms or genetically modified derivatives thereof.

“Cellulolytic, cellulosome forming microorganisms” as referred herein relates to those bacteria and fungi forming the extracellular complexes (called cellulosomes), wherein enzymes are bound via cohesin-dockerin interaction to the backbone scaffold. Further cellulolytic, cellulosome forming microorganisms which may be used in the present invention are: bacteria, such as Acetivibrio cellulolyticus, Bacterioides cellulosolvens, Butyrivibrio fibrisolvens, Clostridium acetobutylicum, C. cellulolyticum, C. cellulovorans, C. cellobioparum, C. josui, C. papyrosolvens, C. thermocellum, C. sp C7, C. sp P-1, Fibrobacter succinogenes, Ruminococcus albus, R. flavefaciens, and fungal microorganisms, such as Piromyces sp. E2.

In a further embodiment the backbone scaffold is derived from the non-catalytic scaffolding protein CipA from Clostridium thermocellum or genetically modified derivatives thereof.

The term “genetically modified derivative” as used herein means that the backbone scaffold protein of the complex of the invention is genetically modified, for example the backbone scaffold is a genetically modified derivative derived from the CipA-protein of C. thermocellum or the backbone scaffold is genetically fused to a dockerin module or a His-tag sequence, or the number or order of the naturally occurring modules (in CipA) is changed, or the nucleotide sequence is changed to introduce or eliminate restriction sites, to adapt the codon usage or to change amino acid residues in certain positions.

In a preferred embodiment the backbone scaffold of the complex of the invention comprises CBM-c1-c1-d3 as shown in SEQ ID NO: 24, c3-c1-c1-d2 as shown in SEQ ID NO: 22, c2-c1-c1 as shown in SEQ ID NO: 26, or derivatives thereof having more than 60% amino acid sequence identity in their cohesin modules.

The term “sequence identity” known to the person skilled in the art designates the degree of relatedness between two or more nucleotide or polypeptide molecules, which is determined by the agreement between the sequences. The percentage “identity” is found from the percentage of identical regions in two or more sequences, taking account of gaps or other sequence features.

The identity of mutually related polypeptides can be determined by means of known procedures. As a rule, special computer programs with algorithms taking account of the special requirements are used. Preferred procedures for the determination of identity firstly generate the greatest agreement between the sequences studied. Computer programs for the determination of the homology between two sequences include, but are not limited to, the GCG program package, including GAP (Devereux J et al., (1984); Genetics Computer Group University of Wisconsin, Madison (WI); BLASTP, BLASTN and FASTA (Altschul S et al., (1990)). The BLAST X program can be obtained from the National Centre for Biotechnology Information (NCBI) and from other sources (BLAST Handbook, Altschul S et al., NCB NLM NIH Bethesda Md. 20894; Altschul S et al., 1990). The well-known Smith Waterman algorithm can also be used for the determination of sequence identity.

Preferred parameters for the sequence comparison include the following:

### Algorithm: Needleman S. B. and Wunsch, C. D. (1970)

Comparison matrix: BLOSUM62 from Henikoff S, and Henikoff J. G. (1992).
Gap penalty: 12Gap-length penalty: 2

The GAP program is also suitable for use with the above parameters. The above parameters are the standard parameters (default parameters) for amino acid sequence comparisons, in which gaps at the ends do not decrease the identity value. With very small sequences compared to the reference sequence, it can further be necessary to increase the expectancy value to up to 100,000 and in some cases to reduce the word length (word size) to down to 2.

Further model algorithms, gap opening penalties, gap extension penalties and comparison matrices including those named in the Program Handbook, Wisconsin Package, Version 9, September 1997, can be used. The choice will depend on the comparison to be performed and further on whether the comparison is performed between sequence pairs, where GAP or Best Fit are preferred, or between one sequence and a large sequence database, where FASTA or BLAST are preferred. An agreement of 60% determined with the aforesaid algorithms is described as 60% identity. The same applies for higher degrees of identity.

In preferred embodiments, the variants according to the invention the derivatives have more than 60% amino acid sequence identity, preferably more than 70%, more preferably more than 80% or 90% amino acid sequence identity in their cohesin modules.

In a further embodiment of the invention, the backbone scaffold of the complex of the invention comprises a carbohydrate binding module (CBM). Preferably, the carbohydrate binding module is a carbohydrate binding module of family CBM3 according to the classification by the CAZy data base (http://www.cazy.oro/Carbohydrate-Binding-Modules.html) from the cipA gene of Clostridium thermocellum that is integrated into or attached to the linear backbone scaffold.

The term “carbohydrate binding module (CBM)” as used herein refers to a contiguous amino acid sequence having carbohydrate binding activity. The CBM may either be introduced into the complex (“mini-scaffoldins”) of the invention or the CBM is present in the enzyme component, or alternatively may be genetically bound to the mini-scaffoldins via fusion to a protein component. Different CBMs may recognize different polysaccharides or polysaccharide structures. The CBM may also be bound to the mini-scaffoldins by genetic modification or chemical reaction with a functional group of the backbone scaffold. The CBM elicits a “targeting effect”, i.e. enhancement of binding between complex and substrate, which is particularly advantageous for insoluble substrates. CBMs are defined as a discretely folded non-catalytic polypeptide module, binding to a polysaccharide or a complex carbohydrate. They can be found or genetically engineered to be modularly fused to enzymes or scaffolding proteins, or as a genetic fusion bound to a backbone scaffold via cohesin-dockerin interaction or other means. In the preferred embodiment they bind to crystalline cellulose and belong to CBM family 3 (CBM3). (see: http://www.cazv.ora/Carbohydrate-Binding-Modules.html) The binding to a polysaccharide can among other methods be determined by retardation of the protein in native gel electrophoresis in which the polysaccharide is homogeneously distributed in the gel.

In a further embodiment, the invention relates to a complex as defined herein, wherein the enzyme component comprises at least a dockerin module and a catalytic module of an enzyme.

The term “module” describes a separately folding moiety within a polypeptide which can be used in a “Lego” like fashion to assemble proteins with new characteristics by genetic engineering or natural recombination. The “catalytic module of an enzyme” as used herein refers to a protein module which contributes the catalytic activity to a polypeptide. All enzymes of the cellulosome are multimodular enzymes and consist of catalytic and non-catalytic modules, at least of a catalytic module and a dockerin module. A non-catalytic module may be a dockerin, cohesin, CBM, S-layer homologous module, or a module with yet unknown function (often called X-module).

In a further embodiment, the invention relates a complex as defined herein, wherein the enzyme components are selected from the group consisting of: processive or non-processive endo-β-1,4-glucanases, processive exo-β-1,4-glucanases and glycosidases from polysaccharolytic or saccharolytic microorganisms or genetically modified derivatives thereof.

The enzyme components combined in the complex of the invention comprise inter alia cellulases from the cellulosome of Clostridium thermocellum, for example the components CbhA, CelA, CelE, CelJ, CelK, CeLR, CelS or CelT of the thermophilic bacterium Clostridium thermocellum, or thermostable β-glycosidases, for example β-glucosidase BglB from the thermophilic bacterium Thermotoga neapolitana.

Exchanging by other activities, taking out or adding enzyme components or changing their molar ratio can extend or enhance the activity of the complexes for other substrates. The new components may include β-glucosidases, hemicellulases (xylanases, mannanases, arabinofuranosidases, glucuronidases, xylan-esterases etc.), pectinases, pectin lyases, amylases and other enzymes for lignocellulosic biomass hydrolysis, other polysaccharides, or the combination of enzymes for a biochemical synthesis pathway.

“Polysaccharolytic microorganisms” as used herein refer to hydrolytic microorganisms capable of degrading polysaccharides, such as amylolytic, pectinolytic, cellulolytic or hemicellulolytic microorganisms. “Saccharolytic microorganisms” as used herein refer to microorganisms using carbohydrates as primary source of carbon and energy.

In a further embodiment, the invention relates a complex as defined herein, wherein the at least three enzyme components are selected from the group consisting of cellulolytic and hemicellulolytic enzymes from other microorganisms.

Examples for polysaccharides are acetan, agar-agar, alginate, amylopectin, arabinan, arabinogalactan, arabinoxylan, carboxymethyl cellulose, cellulose, chitin, chitosan, chrysolaminarin, curdlan, cyclosophoran, dextran, dextrin, emulsan, fructan, galactan, galactomannan, gellan, α-glucan, β-glucan, glucuronan, glucuronoxylan, glycogen, N-acetyl-heparosan, hydroxyethyl cellulose, indican, inulin, kefiran, laminarin, lentinan, levan, lichenin, lichenan, lupin, mannan, pachyman, pectic galactan, pectin, pentosan, pleuran, polygalacturonic acid, pullulan, rhamnogalacturonan, schizophyllan, scleroglucan, starch, succinoglycan, welan, xanthan, xyloglucan, zymosan.

Examples of cellulolytic microorganisms are bacteria such as Acetivibrio cellulolyticus, A. cellulosolvens, Anaerocellum thermophilum, Bacteroides cellulosolvens, Butyrivibrio fibrisolvens, Caldicellulosiruptor saccharolyticus, Cs. lactoaceticus, Cs. kristjansonii, Clostridium acetobutylicum, C. aldrichii, C. celerescens, C. cellobioparum, C. cellulofermentas, C. cellulolyticum, C. cellulosi, C. cellulovorans, C. chartatabidum, C. herbivorans, C. hungatei, C. josui, C. papyrosolvens, C. sp C7, C. sp P-1, C. stercorarium, C. thermocellum, C. thermocopriae, C. thermopapyrolyticum, Fibrobacter succinogenes, Eubacterium cellulolyticum, Ruminococcus albus, R. flavefaciens, R. succinogenes, Achromobacter piechaudii, Actinoplanes aurantiaca, Bacillus circulans, Bacillus megaterium, Bacillus pumilus, Caldibacillus cellulovorans, Cellulomonas biazotea, Cm. cartae, Cm. cellasea, Cm. cellulans, Cm. fimi, Cm. flavigena, Cm. gelida, Cm. iranensis, Cm. persica, Cm. uda, Cellvibrio fulvus, Cv. Gilvus, Cv. Mixtus, Cv. vulgaris, Curtobacterium falcumfaciens, Cytophaga sp., Flavobacterium johnsoniae, Microbispora bispora, Micromonospora melonosporea, Myxobacter sp. AL-1, Pseudomonas fluorescens, Ps. mendocina, Streptomyces alboguseolus, Sm. antibioticus, Sm. aureofaciens, Sm. cellulolyticus, Sm. flavogriseus, Sm. lividans, Sm. nitrosporeus, Sm. olivochromogenes, Sm. reticuli, Sm. rochei, Sm. thermovulgaris, Sm. viridosporus, Sporocytophaga myxcoccoides, Thermoactinomyces sp. XY, Thermobifida alba, Tb. cellulolytica, Tb. fusca, Thermonospora curvata, Xanthomonas sp.; fungi, such as Anaeromyces mucronatus, Aspergillus niger, Caesomyces comunis, Cyllamyces aberensis, Hypocrea sp., Neocaffimastix frontalis, Orpinomyces sp., Phanerochaete chrysosporium, Piptoporus cinnabarinus, Piromyces sp., Piromyces equi, Piromyces sp. E2, Rhizopus stolonifer, Serpula lacrymans, Sporotrichum pulverulentum, Trichoderma (=Hypocrea) harzianum, T. koningii, T. longibrachiatum, T. pseudokoninii, T. reesei, T. viride.

In a further embodiment, the complex of the invention comprises at least three enzyme components derived from dockerin containing components of Clostridium thermocellum or from components of Thermotoga maritima having dockerin fused thereto. The complex of the invention may also comprise dockerin containing enzyme components or enzyme components having dockerin fused thereto from other bacteria.

In a preferred embodiment, the enzyme components comprise CelK-d1 as shown in SEQ ID NO: 8, CelR-d1 as shown in SEQ ID NO: 10, CelT-d1 as shown in SEQ ID NO: 14, CelE-d1 as shown in SEQ ID NO: 16, CelS-d1 as shown in SEQ ID NO: 6 and BglB-d1 as shown in SEQ ID NO: 4, or derivatives thereof or related genes from other bacteria having more than 50%, preferably more than 60%, more than 70%, more preferably more than 80%, more preferably more than 90%, and most preferred more than 97% amino acid sequence identity.

In a further embodiment, the complex of the invention comprises a backbone scaffold comprising the proteins CBM-c1-c1-d3 as shown in SEQ ID NO: 24, c3-c1-c1-d2 as shown in SEQ ID NO: 22, c2-c1-c1 as shown in SEQ ID NO: 26 and the enzyme components comprising CelK-d1 as shown in SEQ ID NO: 8, CelR-d1 as shown in SEQ ID NO: 10, CelT-d1 as shown in SEQ ID NO: 14, CelE-d1 as shown in SEQ ID NO: 16, CelS-d1 as shown in SEQ ID NO: 6 and BglB-d1 as shown in SEQ ID NO: 4.

The invention further provides a method for preparing the complex as defined herein comprising the steps:

a) recombinantly producing the at least three enzyme components as defined herein,
b) recombinantly producing the backbone scaffold of any one of claims 1 to 8,
c) mixing the purified, partially purified or non-purified components of a) and b) in vitro; and
d) randomly binding the enzyme components to the backbone scaffold.

In a further embodiment, the method for producing the complex as defined herein comprises the step of binding the recombinantly produced backbone scaffolds or enzyme particles to a carrier particle. Suitable particles include nano-particles as defined herein above, preferably polystyrene coated nano-particles, such as a poly-styrene coated ferromagnetic nano-particle, or chemically functionalized superparamagnetic nano-particles or other small nano-particles, preferably with a diameter of 10 to 2000 nm, more preferably 30 to 250 nm, with carboxyl or amino groups attached to the surface. State-of-the-art coupling chemistry is used for binding backbone scaffolds or the enzymes to the particle surface.

The recombinant production of the enzyme components can be performed by gene cloning and modification techniques well known in the art, for example the engineered enzyme components may be fused to dockerin, cohesin and/or other non-catalytic modules, optionally followed by protein engineering of the components to enhance recombinant production, for example by optimizing the secretion signals, changing protein segments decreasing successful expression or secretion, or the codon usage. In a further step the backbone scaffolds (“mini-scaffoldins”) of the invention were recombinantly produced, optionally fused cohesin modules and spontaneously combined to form the complex by mixing the enzyme components and the backbone scaffolds. The components may be purified, partially purified or non-purified, preferably partially purified or non-purified. Preferably, the molar ratio of cohesin and dockerin modules in the enzyme-backbone scaffold mixture is 1:1. The at least three enzyme components are mixed together in vitro in a molar ratio of 1:1 to 1:50, 1:1.5 to 1:30, preferably 1:1.8 to 1:15.

The enzyme components thereby randomly bind to the backbone scaffold. The dockerin and cohesin modules of the cohesin-dockerin interaction as defined herein are interchangeable, that means the cohesin modules may either be present in the backbone scaffold or in the enzyme component and the dockerin modules vice versa.

Preferably, at least 3 out of the cellulosomal components, for example the components CbhA, CelA, CelE, CelJ, CelK, CelR, CelS or CelT of the thermophilic bacterium Clostridium thermocellum, or thermostable β-glycosidases, for example β-glucosidase BglB from the thermophilic bacterium Thermotoga neapolitana, are combined on a backbone molecule, preferably in the molar ratio of 0.05 to 1.5 parts each of BglB, CbhA, CelE, CelJ or CelT, in the molar ratio of 0.1 to 3.0 parts each of CelK and CelR, and in the molar ratio of 0.2 to 6.0 each of CelA and CelS. In another embodiment the molar ratio is 0.1 to 1.0 parts for each of BglB, CbhA, CelE, CelJ or CelT, in the molar ratio of 0.2 to 1.0 parts for each of CelK and CelR, and in the molar ratio of 0.5 to 1.0 each of CelA and CelS. In the most preferred embodiment the molar ratio is 0.06 to 0.6 parts each of BglB, CbhA, CelE, CelJ or CelT, in the molar ratio of 0.1 to 1.8 parts each of CelK and CelR, and in the molar ratio of 0.3 to 2.0 each of CelA and CelS.

In a further embodiment, the invention provides a method for preparing the complex of the invention as defined herein above, wherein the total amount of backbone scaffolds in step c) and the total amount of enzyme components are mixed together in a molar ratio of 1 cohesin module to 1 enzyme component, and the at least three enzyme components are mixed together in vitro in a molar ratio of 1:1 to 1:50, 1:1.5 to 1:30, preferably 1:1.8 to 1:15 preferably 1:1 to 1:15 to each other.

In a further embodiment, the invention provides a complex produced by the method described herein.

The “molar ratio” as far as it relates to the ratio of backbone scaffolds to enzyme components is calculated as molar ratio of 1 binding site, preferably a cohesin module comprised in the backbone scaffold to 1 binding site, preferably a dockerin module comprised in the enzyme component.

In a further embodiment, the invention provides a method for enzymatic hydrolysis of polysaccharide substrates comprising the steps of:

a) mixing the complex of the invention with insoluble cellulose; and optionally b) isolating the degradation products.
Mixing the complex of the invention with insoluble cellulose is preferably performed in water environment at the optimal or near the optimal pH and temperature. The optimal or near optimal pH is 6.5±0.5. The optimal temperature is in the range of 25-65° C., preferably 30-65° C.; most preferred about 55° C.

In a further embodiment, the invention provides the use of the complex of the invention for enzymatic hydrolysis of polysaccharide substrates.

In a preferred embodiment of the invention, the polysaccharide substrate as described herein above is crystalline cellulose or a crystalline cellulose containing substrate.

### BRIEF DESCRIPTION OF THE DRAWINGS

FIG. 1 shows colonies of a selected non-adsorbing culture of C. thermocellum. On a turbid agar surface colonies surrounded by a dark (=cleared) halo of hydrolyzed cellulose are visible. The ruler shows 1 and 2 cm markings.

FIG. 2 illustrates the approximate 3D-structure (curtesy H. Gilbert) of the recombinant scaffoldin constructs. Derivatives of the CipA-protein of C. thermocellum (top row: c1=cohesin 1 etc.; CBM=carbohydrate binding module)

FIG. 3 shows the specific activity [mU/mg as glucose equivalents] of complexes with native soluble cellulosomal components from the mutant SM1 (SM901) and mini-scaffoldins as well as complete native scaffoldin CipA of C. thermocellum. Control: native cellulosome from C. thermocellum. Determination of activity on 0.5% Avicel (T=60° C., pH 6.0). (Coh: Cohesin, CBM: Carbohydrate-Binding-Module).

FIG. 4 illustrates the nanoparticle-linker scaffoldin-cellulase complex (NLSC). For simplicity the scaffoldin is shown with only 2 cohesins (instead of 9). The nanoparticles and the various molecules are not drawn to scale.

FIG. 5 shows the activity of various complexes with and without nanoparticle-binding. SM901: the mutant cellulosomal components without scaffoldin.

FIG. 6 compares pH-stability of free enzymes (SM901) and enzymes bound to nanoparticles (NP+SM901).

FIG. 7 compares the temperature stability of free enzymes (SM901) and enzymes bound to nanoparticles (NP+SM901).

FIG. 8 shows a thin layer chromatography of the hydrolysis products from soluble (CMC) and insoluble (crystalline) cellulose (Avicel) as substrate. A comparison of soluble enzymes (SM901), artificial complexes and native cellulosome is shown.

FIG. 9 shows the activity of various mixtures of recombinant cellulosomal components with scaffolding protein. Crystalline cellulose is used as substrate.

FIG. 10 shows the specific activity of the soluble cellulosomal components (SM901), the same enzymes in complex form (with scaffoldin), the synthetic mixture with recombinant components (SM901+CipA+Endo+Exo [NTC]), a commercially available Trichoderma reesei enzyme preparation, and the native cellulosome from Clostridium thermocellum. Crystalline cellulose (0.5% w/v) as substrate. Activity calculated as μmol/min as glucose equivalents.

FIG. 11 shows the nucleotide sequence and the amino acid sequence of the backbone scaffolds CBM-c1-c1-d3, c3-c1-c1-d2, c2-c1-c1 and the enzyme components CelK-d1, CelR-d1, CelT-d1, CelE-d1, CelS-d1 and BglB-d1.

FIG. 12 shows a schematic view of a preferred nano-particle.

FIG. 13 shows particles separated from a solution due to their superparamagnetic behavior using a strong disc magnet. The reaction solution can easily be removed with recovery rates well above 93%.

### EXAMPLES

The invention will be further explained by the following Examples, which are intended to be purely exemplarily of the invention, and should not be considered as limiting the invention in any way.

The examples demonstrate the hydrolytic cellulose degradation by a system of 6 recombinantly expressed cellulases which are bound to a protein carrier, a backbone scaffold imitating the scaffoldin CipA (cellulosome integrating protein). The backbone scaffold carries cohesin binding modules which bind tightly and specifically to dockerin modules forming the C-terminus of the enzyme components. This is an in vitro assembled complex resembling the cellulosome of the thermophilic anaerobic bacterium Clostridium thermocellum. The cohesins and dockerins bind each other spontaneously. Such complexes, when reconstituted in vitro with the right components in the correct ratio, exhibit higher activity on crystalline cellulose than the native cellulosomes isolated from the bacterium.

### Example 1

### Isolation of a Non-Cellulosome-Forming Mutant

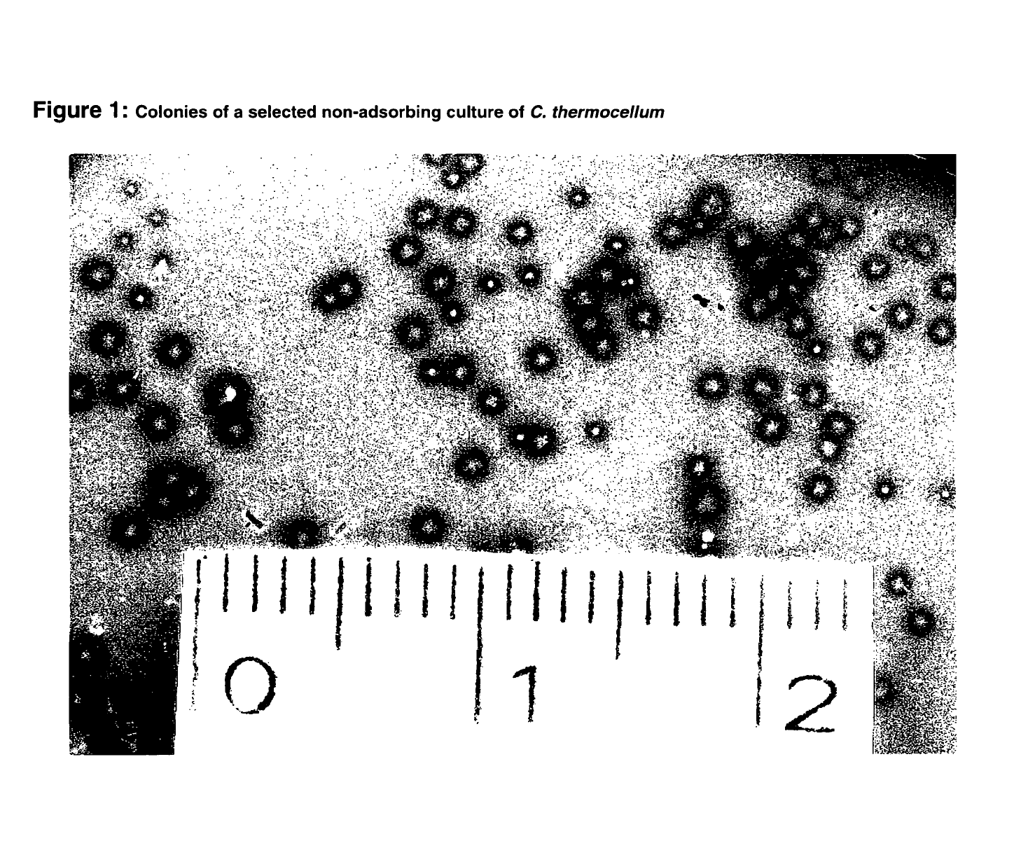

From mutagenized cultures of C. thermocellum mutants were isolated (FIG. 1). Six colonies with a reduced or absent ability to form clear halos in the cellulose around the colonies were randomly selected. One of the C. thermocellum mutants, SM1, had completely lost the ability to produce the scaffoldin protein CipA or an active cohesin. Enzymes from wild type (having cellulosomes) and mutant SM1 (no cellulosomes; free enzymes) were tested on barley β-glucan, CMC (both control), and micro-crystalline cellulose MN300. The enzymatic activity on barley β-glucan and CMC were about 8.5 and 1.0 U mg⁻¹ protein respectively for both strains (Table 1). In contrast, specific activity on crystalline cellulose was dramatically reduced in the mutant SM1, up to 15 fold as compared to the wild type.

The mutant produced the cellulosomal components in approximately equal amounts compared to the wild type, with the exception of the CipA component (the scaffoldin CipA) which was completely missing. Supramolecular complexes were completely missing in the mutant. This indicates an inability of the mutant to properly form cellulosomes. The approximately 50 cellulosomal protein components consequently appeared as dispersed, soluble, non-complexed proteins which are produced in an amount and distribution similar to that observed for the wild type.

TABLE 1
Enzymatic activity of concentrated culture supernatants of the
mutant SM1 and the wildtype on barley β-glucan, CMC (both
soluble) and MN300 cellulose (crystalline).

| | SM1 (U mg⁻¹ protein) | Wt (U mg⁻¹ protein) |
|---|---|---|
| β-Glucan | 7.9 ± 1.1 | 9.5 ± 0.9 |
| CMC | 1.1 ± 0.1 | 1.2 ± 0.1 |
| MN300 (×100) | 0.3 ± 0.1 | 4.4 ± 0.1 |

### Example 2

### Reconstitution of the Cellulosome

### A: Preparation of Enzyme Components

The mutant SM1 and the mutant supernatant proteins (SM901) were selected to reconstitute an artificial cellulosome. In addition, genes coding for cellulase components were cloned and characterized for their biochemical parameters such as pH and temperature optimum and activity on different substrates. The five most prominent enzyme components with cellulase activity were selected from previous data on the composition of the cellulosome¹¹. In addition, β-glucosidases derived from a number of thermophilic saccharolytic bacteria were biochemically characterized. The β-glucosidase BglB from Thermotoga maritima was selected due to its high thermostability and high activity on cellodextrins. The gene was fused to a downstream dockerin module from C. thermocellum cellulase CelA. Optimal expression conditions were determined. The enzymes containing catalytic and non-catalytic modules including a dockerin module formed thereby are herein designated with CelK-d1, CelR-d1, CelT-d1, CelE-d1, CelS-d1 and BglB-d1. The amino acid sequences of CelK-d1, CelR-d1, CelT-d1, CelE-d1, CelS-d1 and BglB-d1 are shown in SEQ ID NO: 4, 6, 8, 10, 14 and 16.

For easier purification they were cloned with an N-terminal His-tag to allow for an easy purification by affinity chromatography. The State of the art technology was used to clone the amplified DNA fragments in frame into a restriction site downstream of a promoter-operator sequence and a 6×His sequence (e.g. pQE vector from Quiagen).

The molar stoichiometry of the components was kept in balance by calculating the number of cohesins and dockerins.

### B: Preparation of the Backbone Scaffolds

The mini-scaffoldins described hereafter were constructed by combining cohesin, dockerin and CBM sequences from the CipA gene of C. thermocellum, from C. thermocellum cellulosomal components and from C. josui. The sequences were optimized for the codon usage of E. coli, i.e. most rare codons for the scarcily expressed tRNA genes argU, ileY, and leuW, which recognize the AGA/AGG, AUA, and CUA codons, were displaced by synonymous codons. The sequences thus derived were synthesized and expressed in E. coli plasmid vectors, such as pQE, according to the art. In one embodiment the cohesins 3-4 (type I) and CBM3 from cipA were used as well as cohesin c3 (type II) and dockerin d3 from olpB of C. thermocellum, or from cipA c2 and dockerin d2 from cellulosomal components of Clostridium josui. The backbone scaffolds used in the Example are designated herein with CBM-c1-c1-d3, c3-c1-c1-d2, c2-c1-c1. The amino acid sequences of CBM-c1-c1-d3, c3-c1-c1-d2, c2-c1-c1 are shown in SEQ ID NO: 22, 24 and 26.

### C: Expression, Purification and Enrichment of Dockerin-Enzyme Components

The enzyme components were initially produced by the mutant SM1. However, C. thermocellum can produce only a limited amount of exoprotein due to energy limitations in its anaerobic life style. Even strain development will not lead to a significant increase in the amount of exoproteins. To replace the native enzyme mixture of more than 30 components by an artificial mixture of cellulases, the major cellulosomal components were prepared from a recombinant host. For experimental simplicity E. coli was used as host. Other bacteria may be better suited for a low cost, high yield production of recombinant proteins. Any industrial producer strain with high yield for a given protein will be appropriate.

The enzyme components were isolated from the recombinant host and purified by His-tag affinity chromatography, or enriched by heat precipitation of E. coli proteins whereby the recombinant proteins remain in the soluble phase. Heat precipitation was performed by heating the protein solution to 65° C. for 10 min and removing the precipitated E. coli proteins by centrifugation according to the art. Enrichment was also successful by ultrafiltration (cutoff 10.000 Dalton).

D: Complexation of Enzyme Components with the Backbone Scaffold

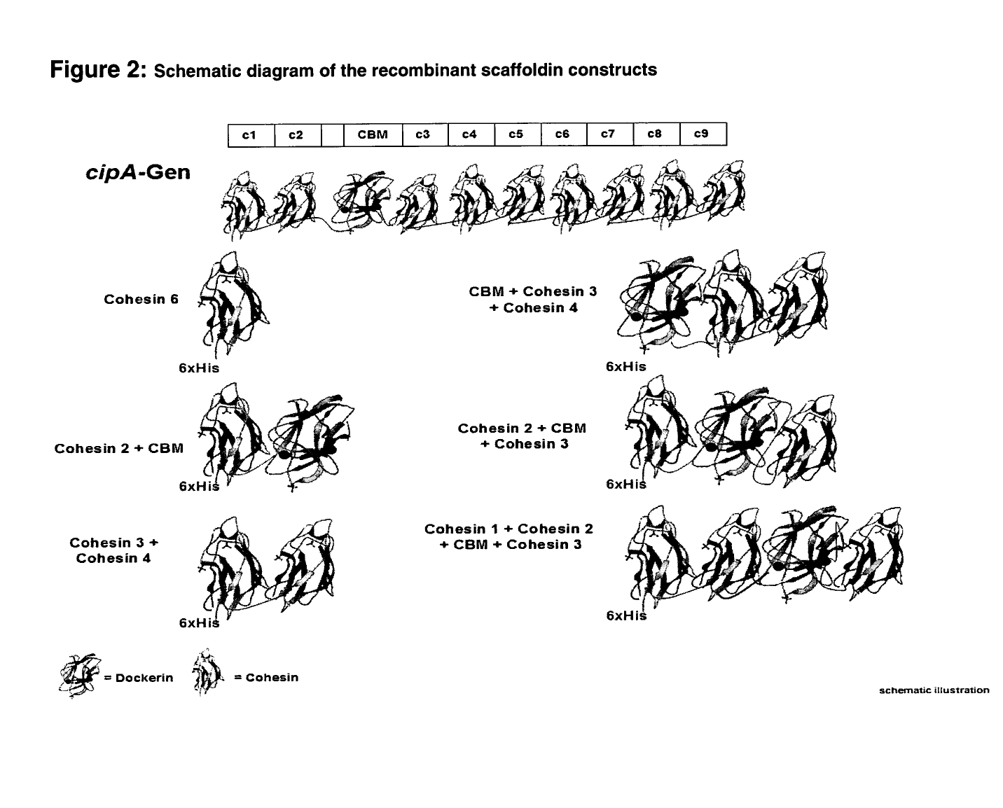

They were then bound to recombinant backbone mini-scaffolds consisting of various cohesins with or without a carbohydrate binding module, for example CBM-c1-c1. The complex can be reconstituted from the components by simple stoichiometric addition to the mixture of enzyme components (one dockerin bearing component per cohesin module present in the mixture) in the presence of calcium, in one embodiment 20 mM CaCl₂. Sample structures of the recombinant mini-scaffoldin constructs are depicted in FIG. 2. Complexation occurs by spontaneously combining the dockerin-enzyme components with mini-scaffoldins consisting of various cohesins via cohesin-dockerin interaction. They were then used to measure the effect of complexation, either with native enzymes or with enzymes isolated from recombinant hosts.

### Example 3

### Activity of the Artificial Cellulosome Complex

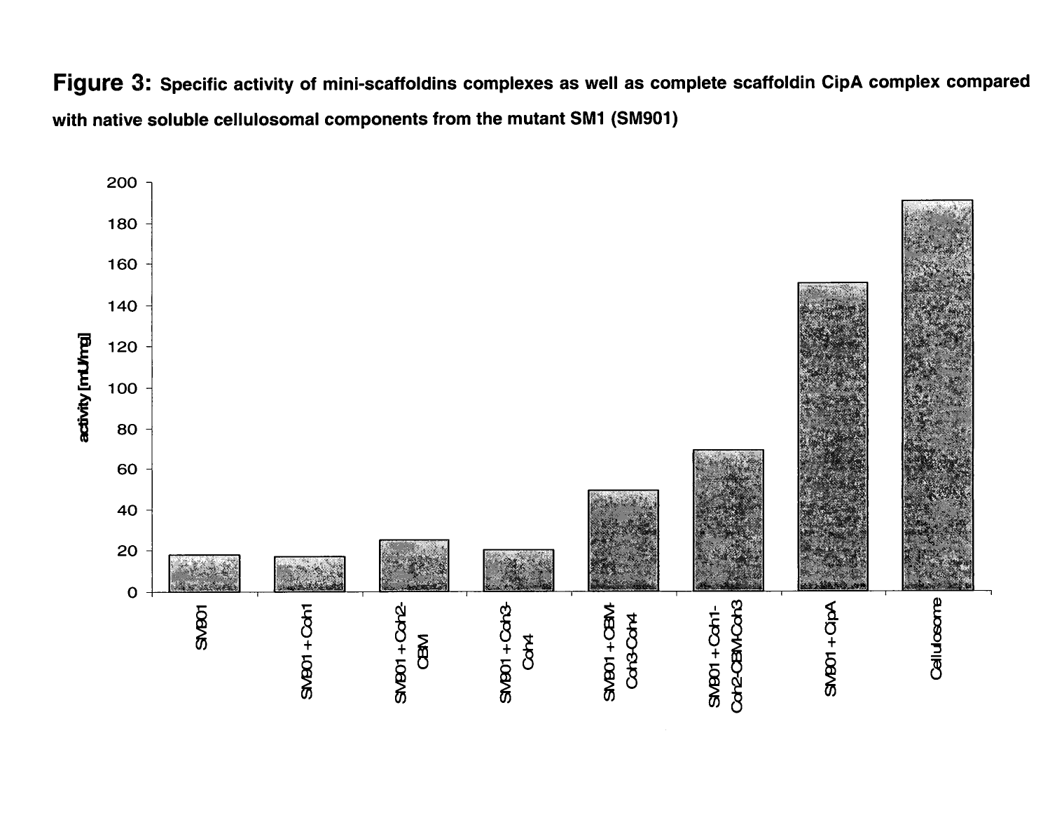

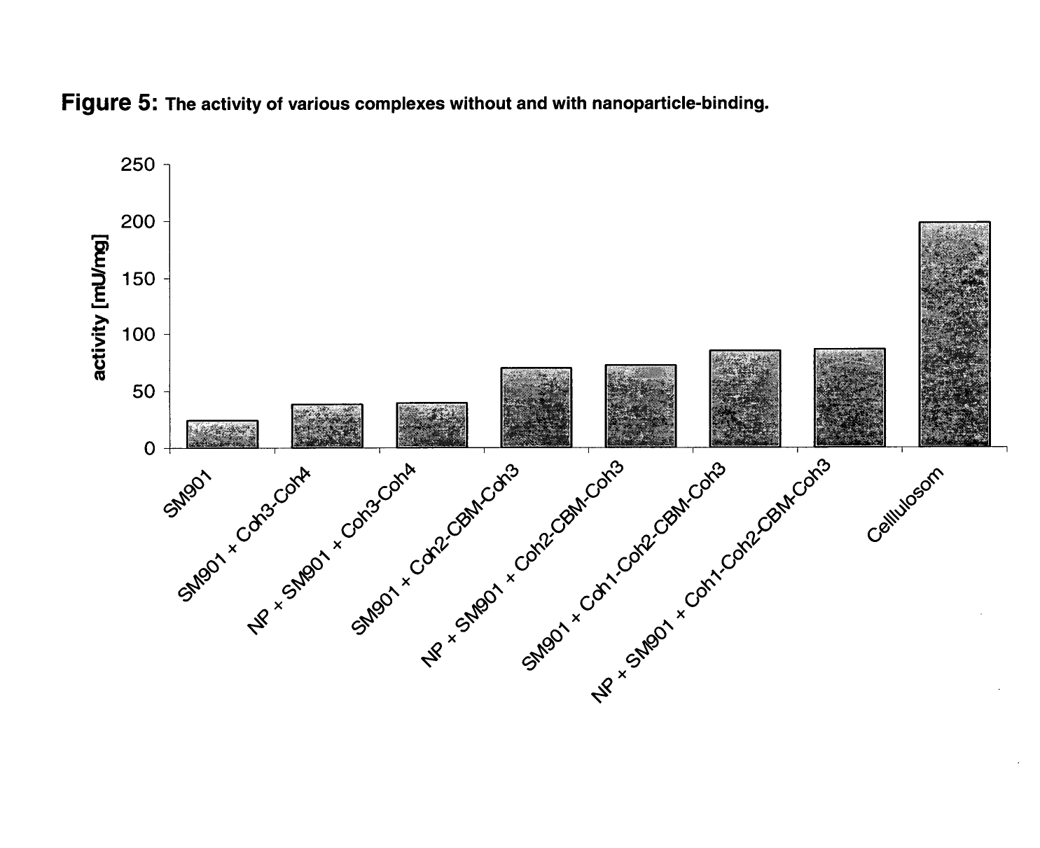

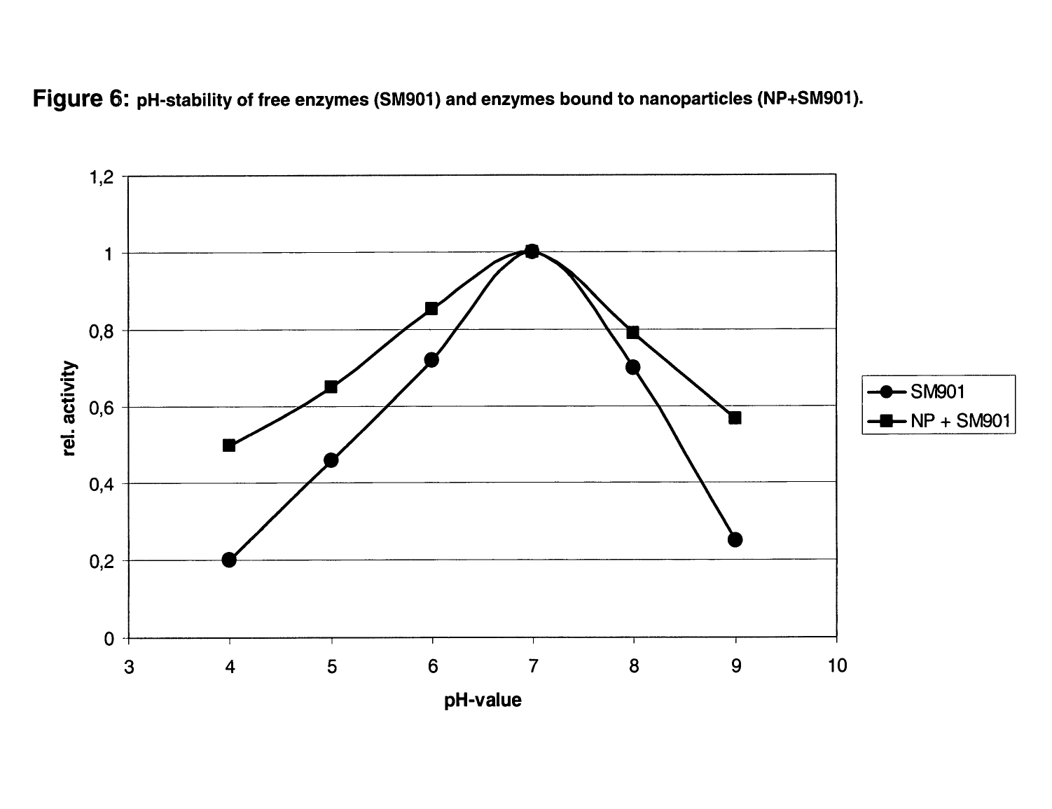

A mixture of such complexes with and without CBMs were now bound via an optimized aliphatic linker molecule on the surface of polystyrene nano-particles. Such structures are schematically shown in FIG. 3. The binding of various mini-scaffoldin complexes on hydrolysis of crystalline cellulose resulted in an increase in activity despite the sterical hindrance and a certain loss of degree of freedom of the enzyme components due to the dense covering of the nanoparticles (FIG. 3, 4). In addition, the pH range of the enzymes was broader if the proteins were bound to the particle (FIG. 5). This was also true for the temperature stability of the cellulases (FIG. 6). Both of these results are an important advantage for technical application.

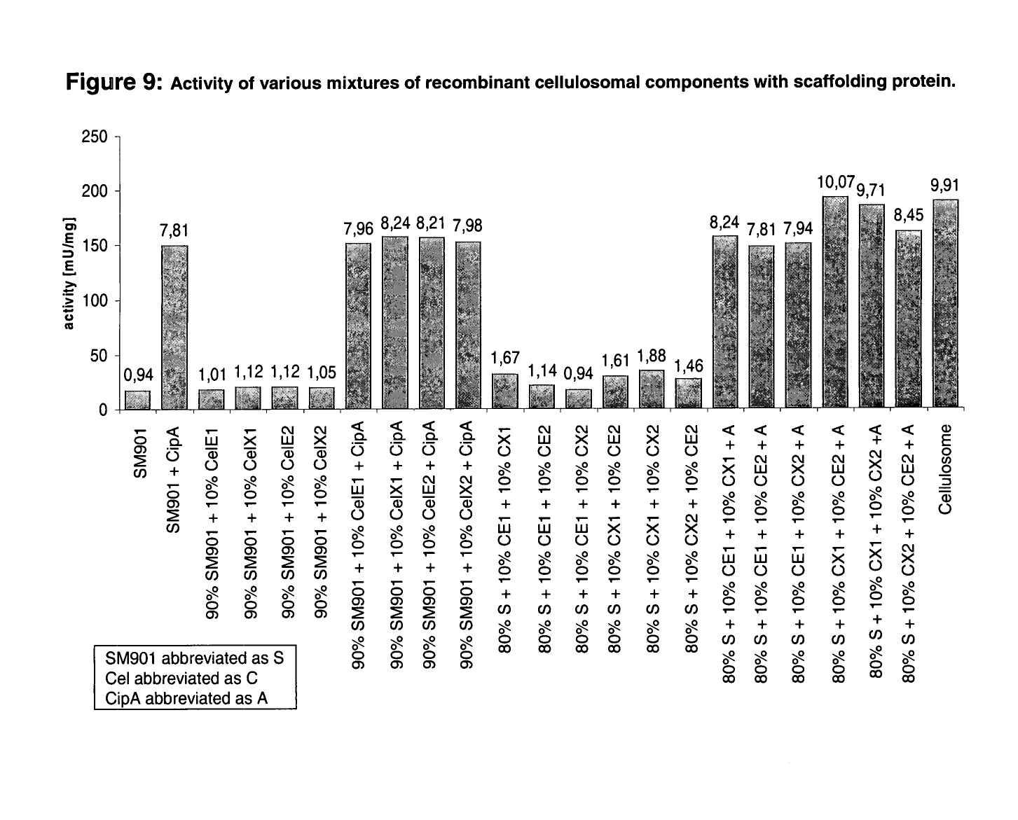

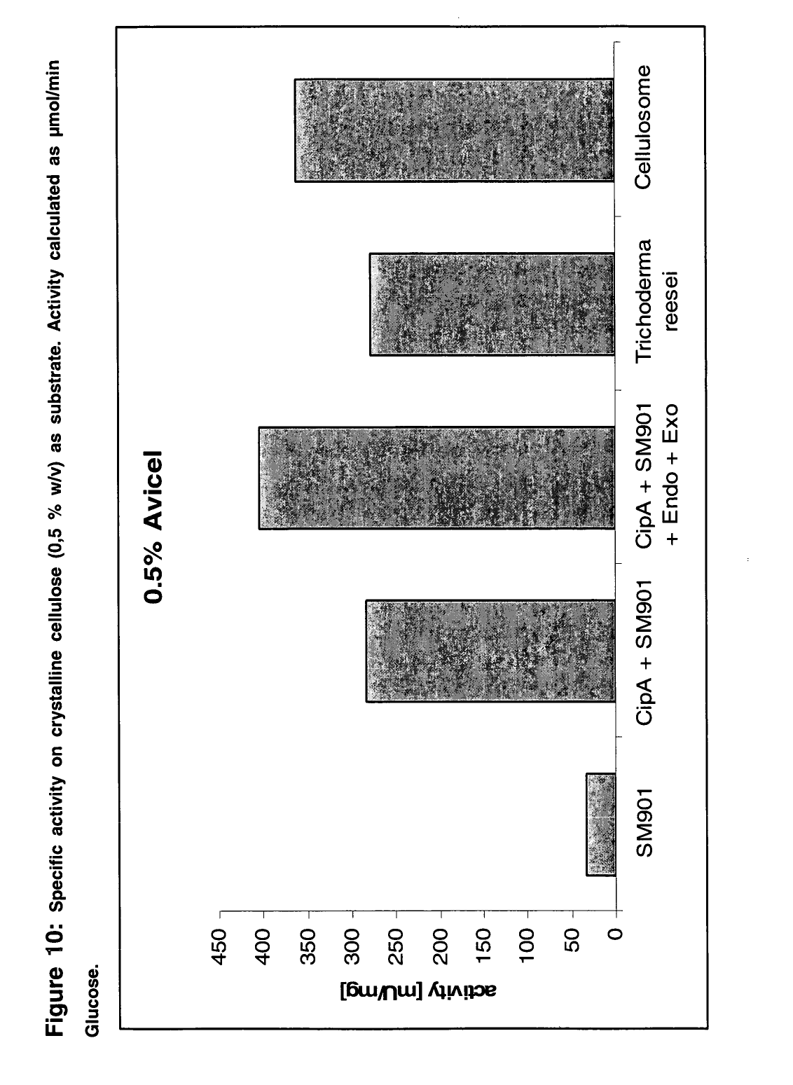

To test the feasibility of that approach, a part of the SM901 component mixture was replaced by one or more of the recombinant cellulases. Despite the decrease of SM901 components in the mixture, the result showed the activity of the complex on crystalline cellulose was slightly increasing when one recombinantly produced component was added. It increased significantly when a mixture of recombinant enzymes was added (FIG. 9). In certain mixtures the activity of synthetic mixtures was higher than that of the native cellulosome. By complete replacement and properly balanced stoichiometry, the synthetic complexes exhibit even higher activity.

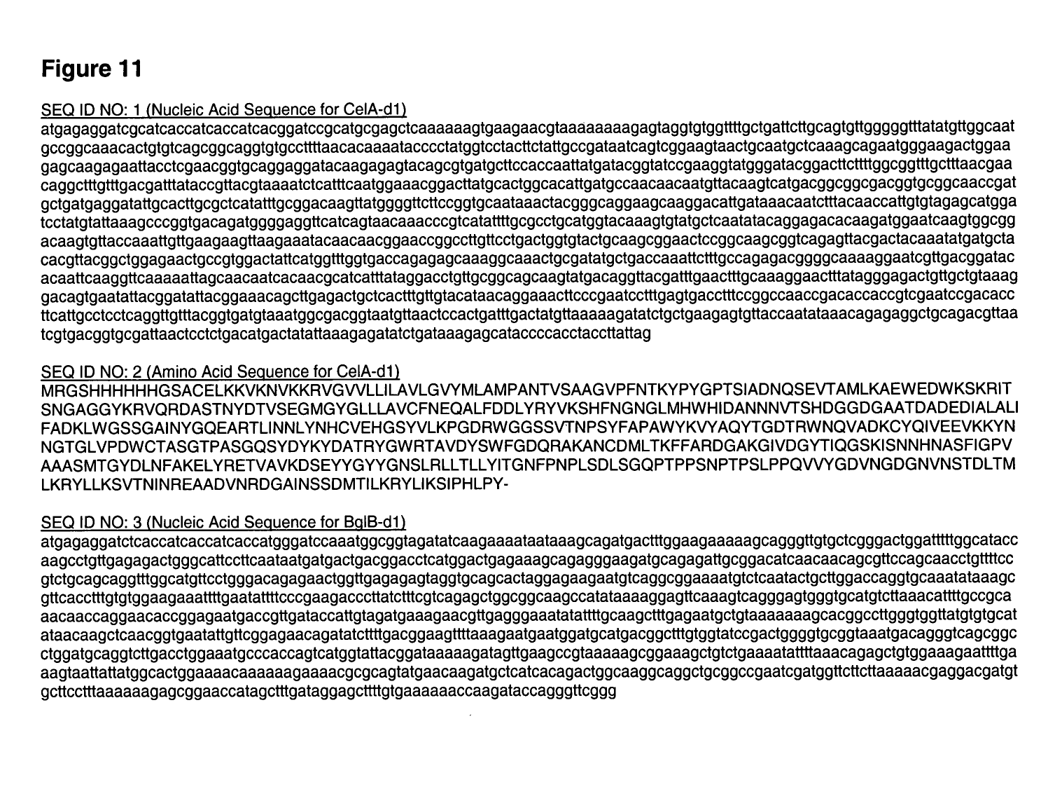

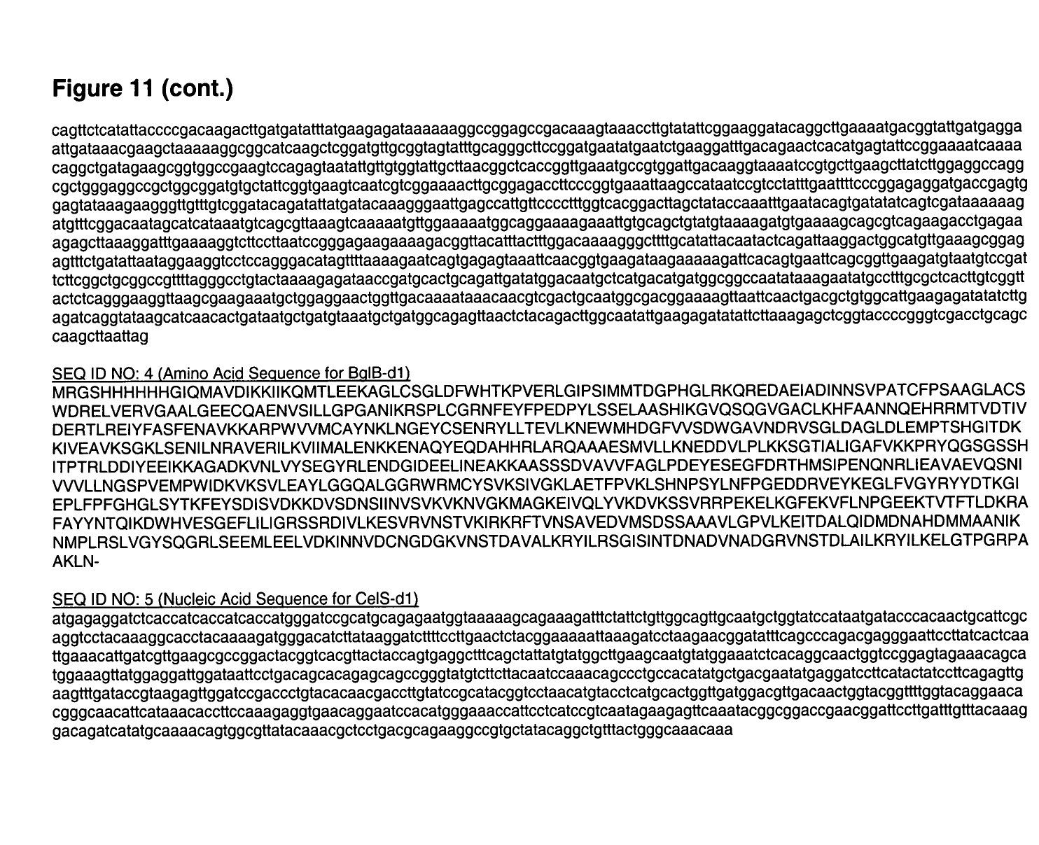

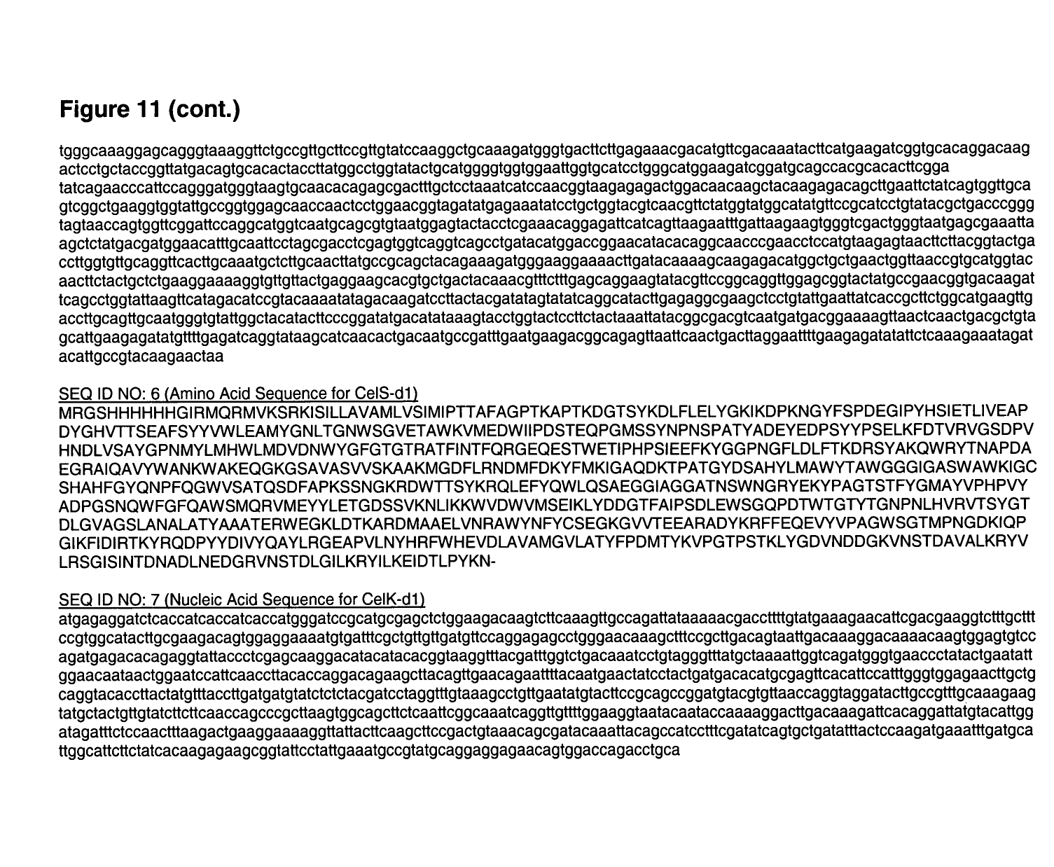

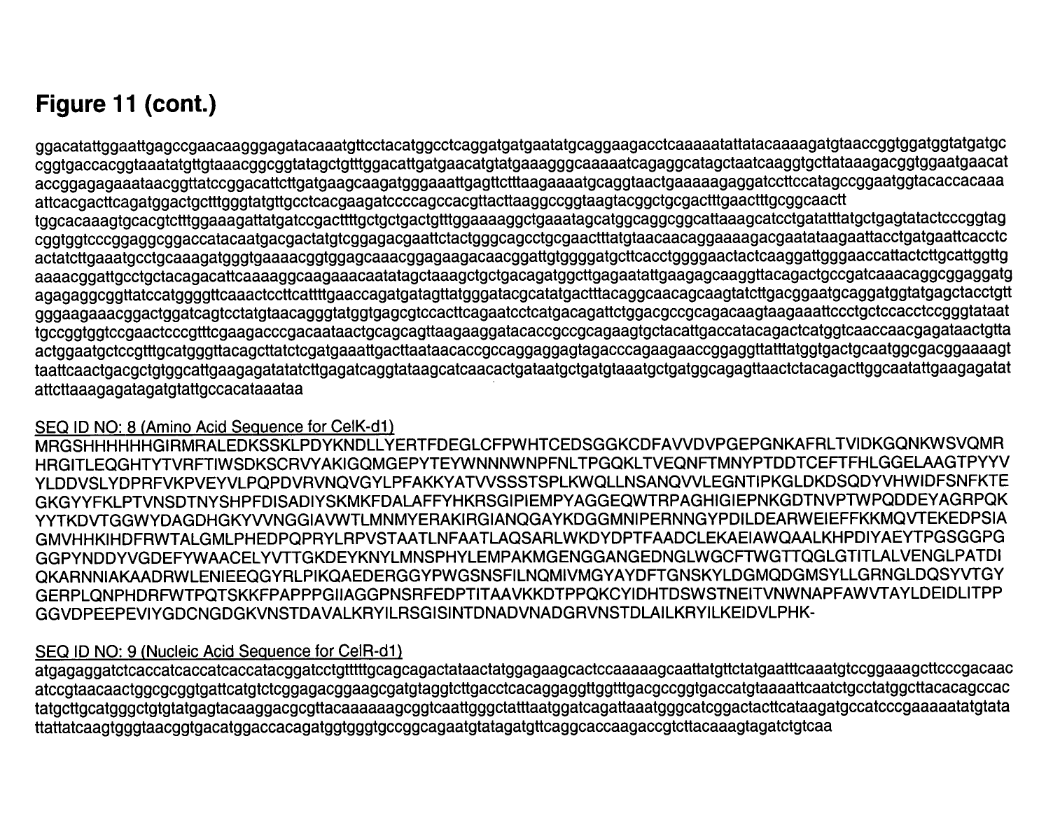

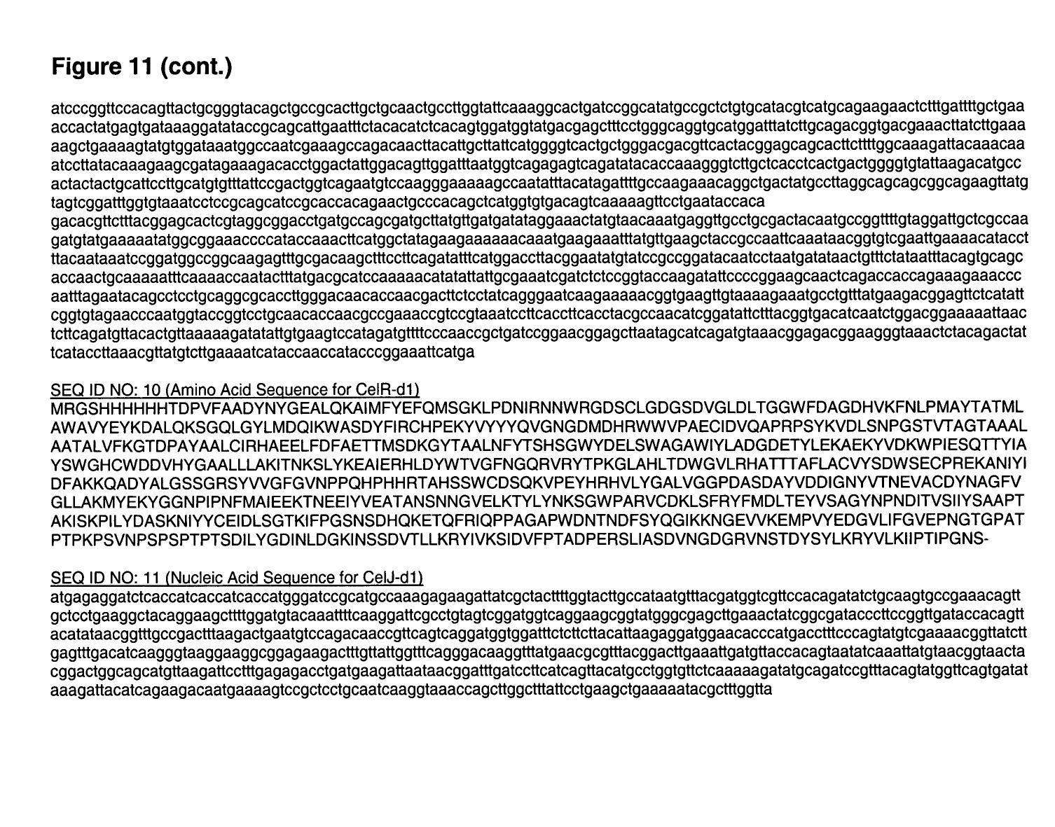

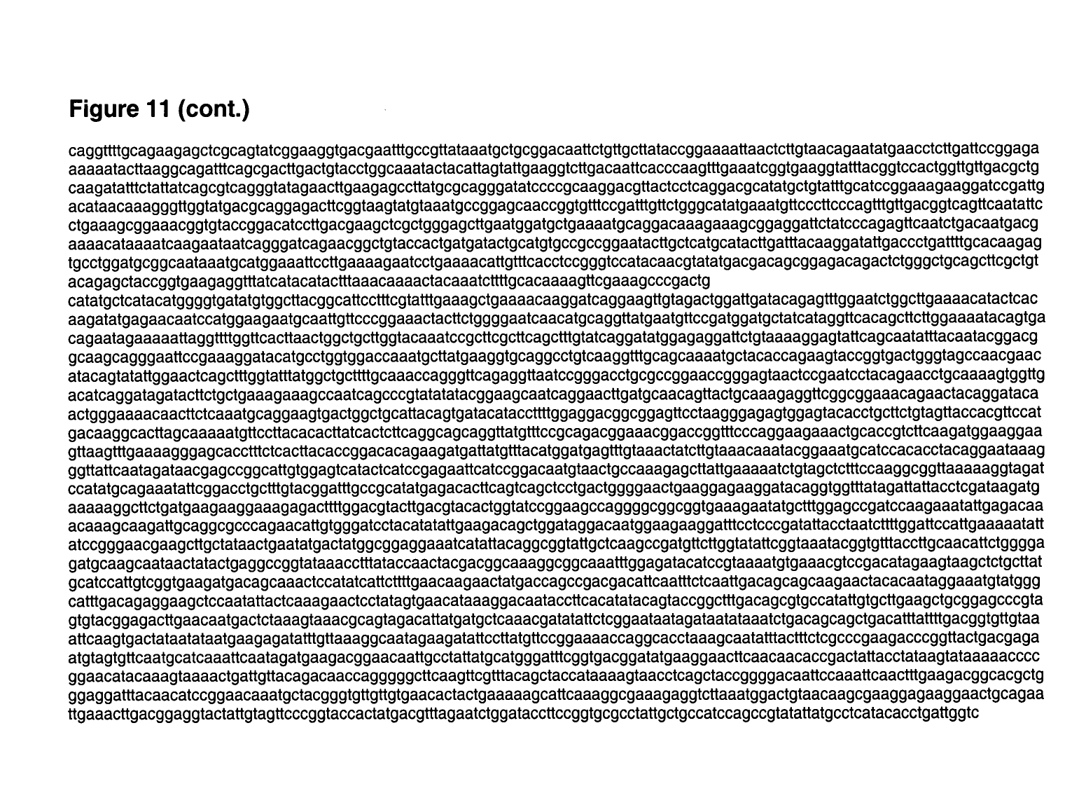

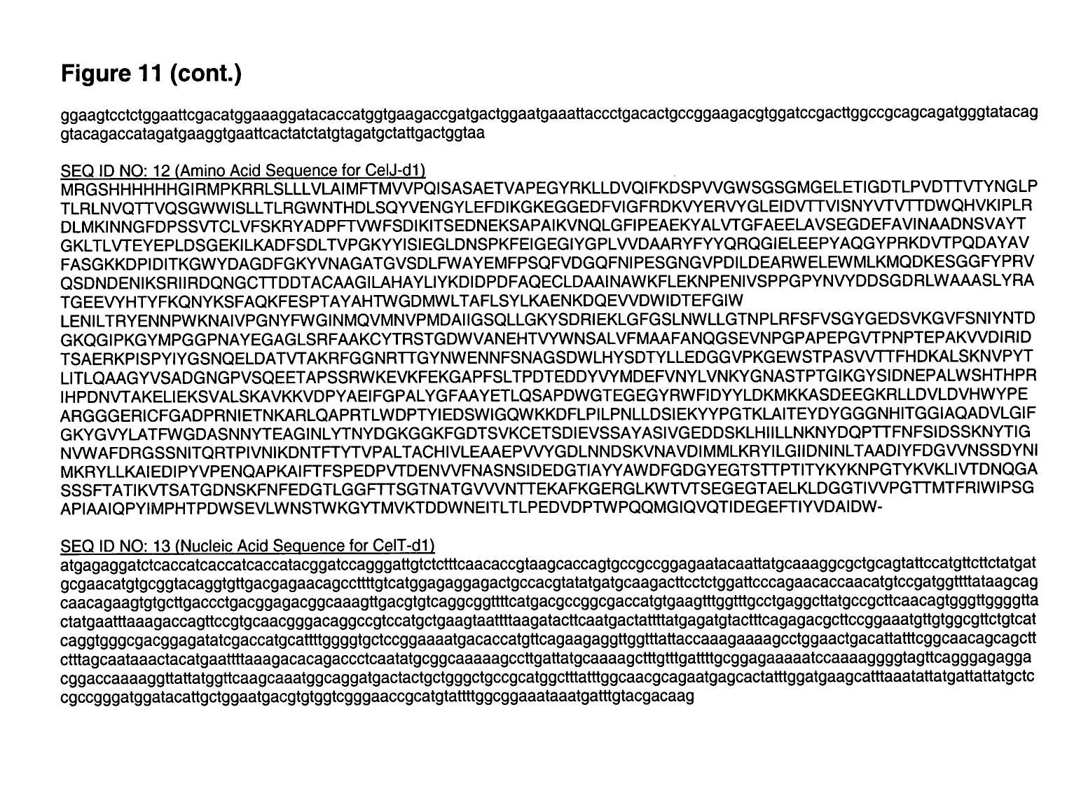

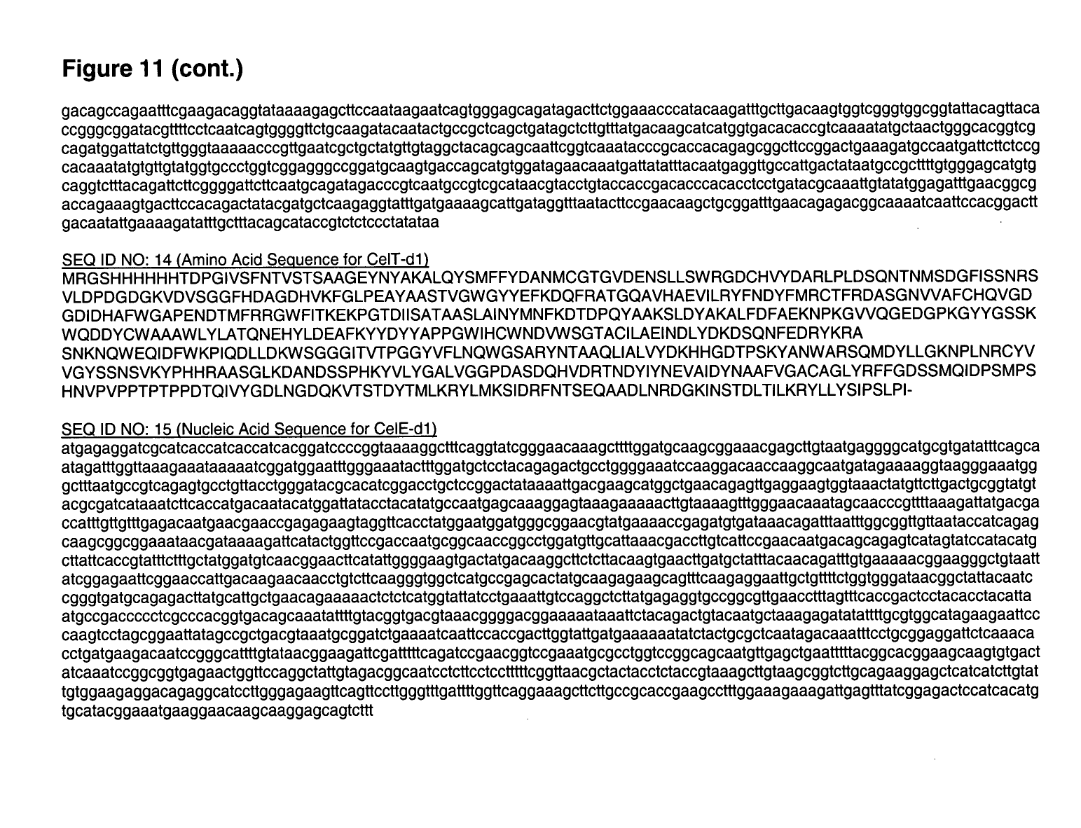

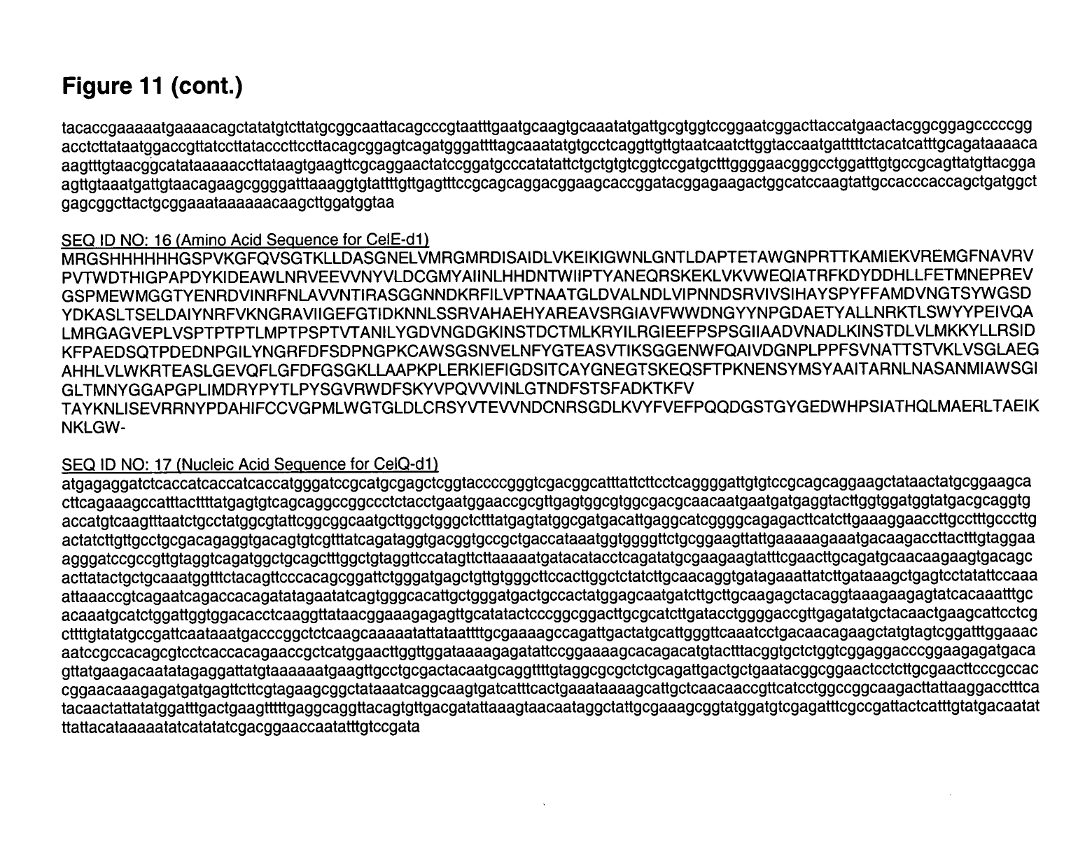

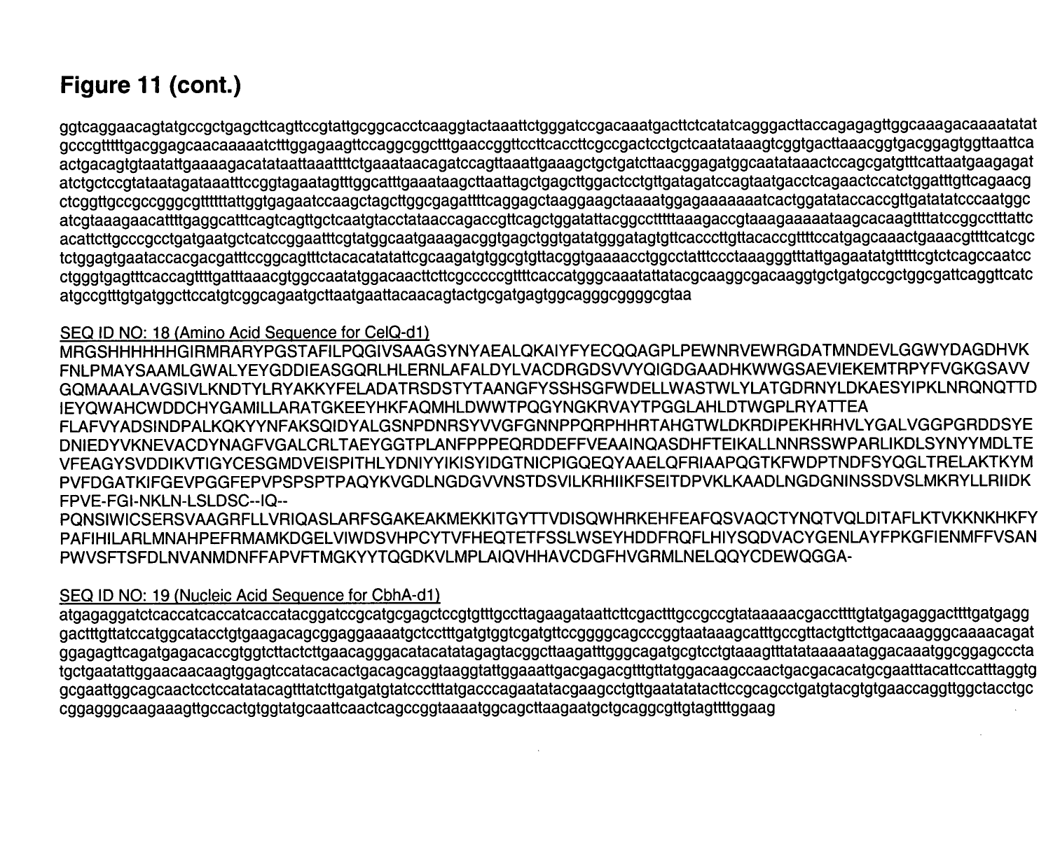

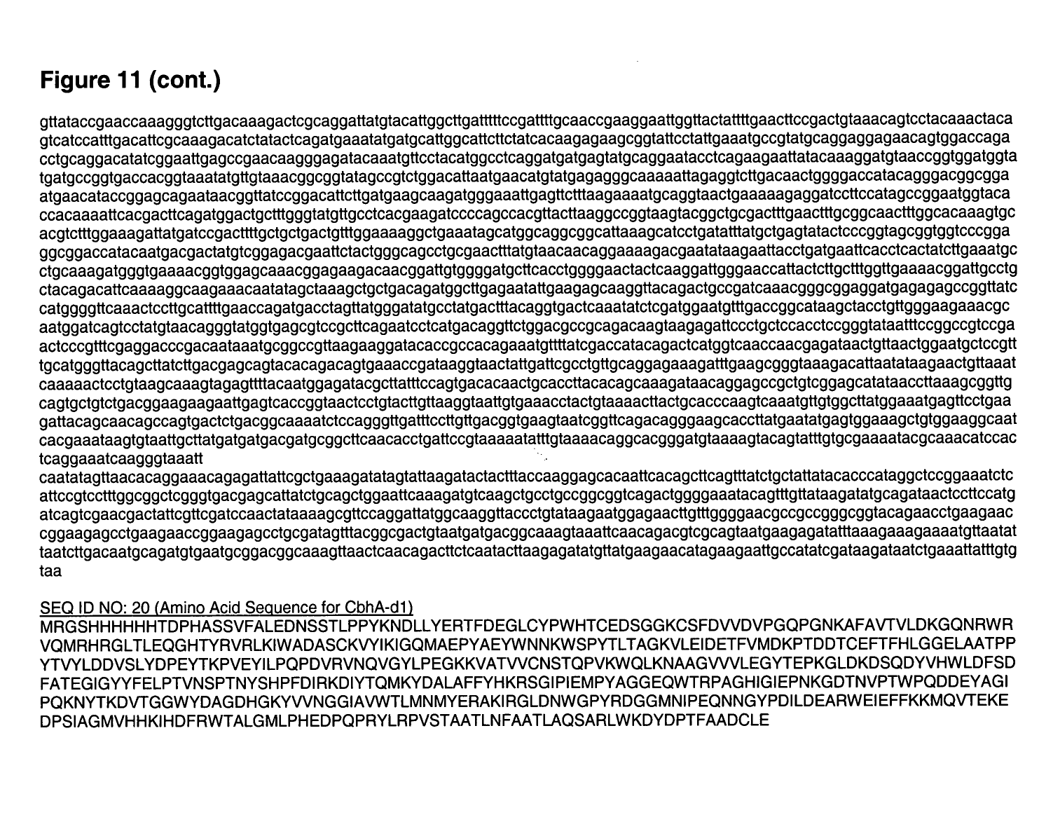

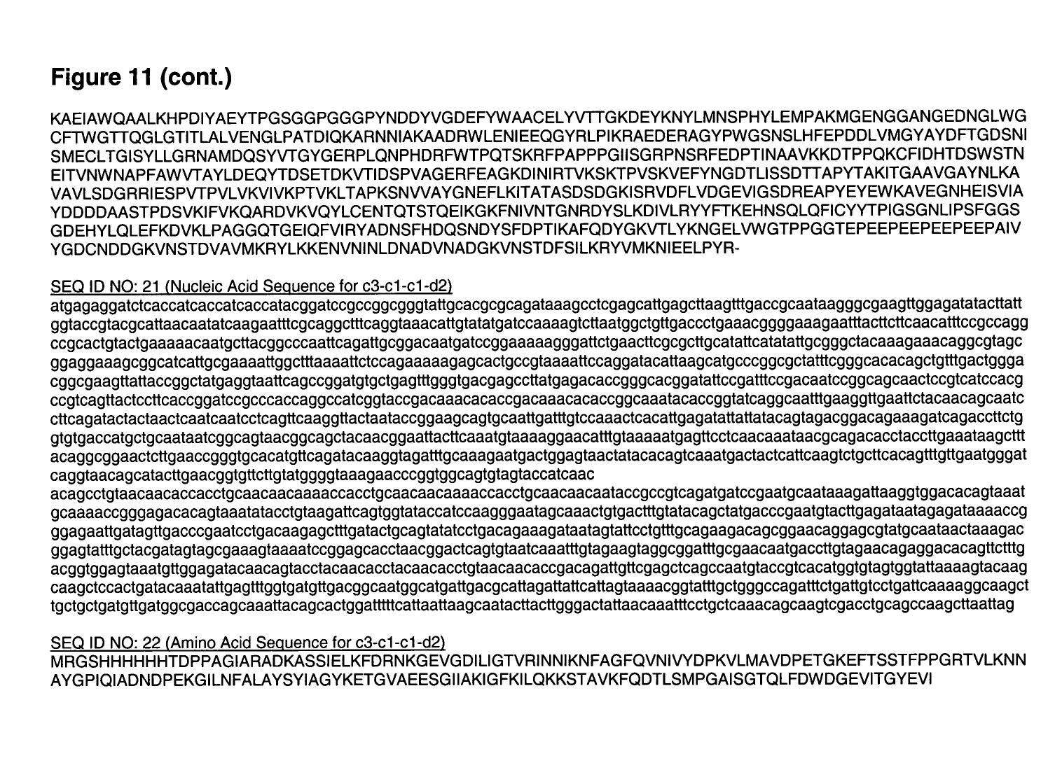

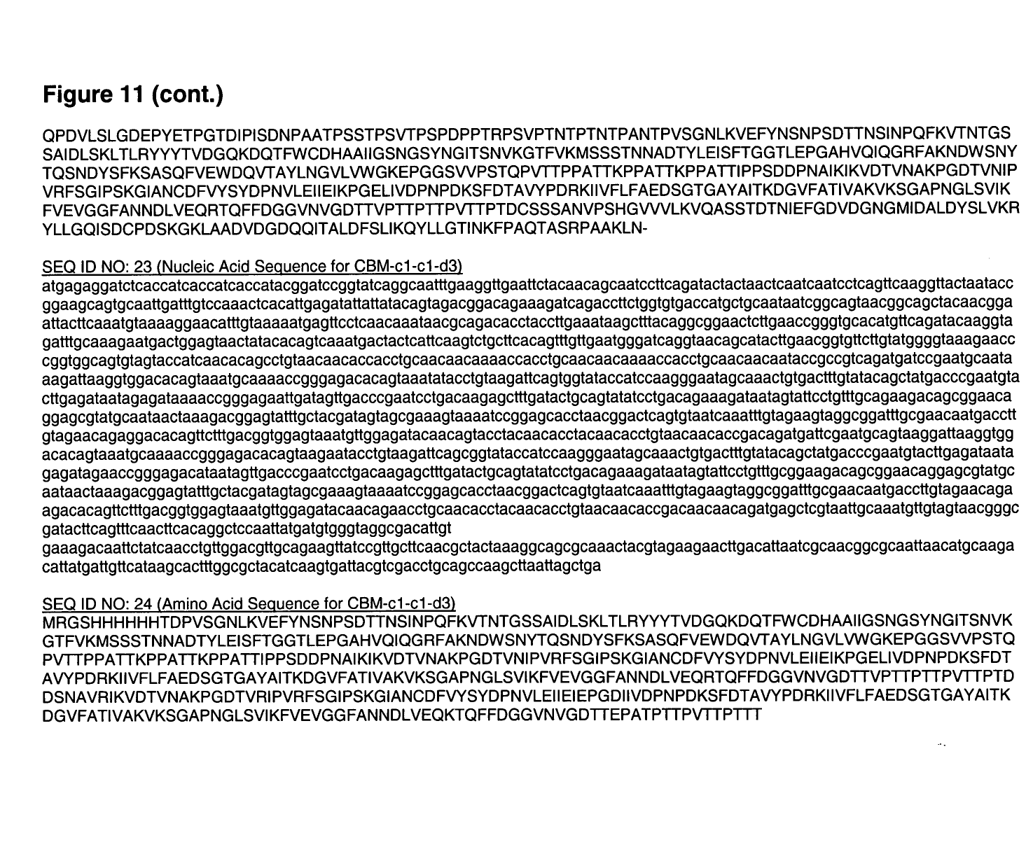

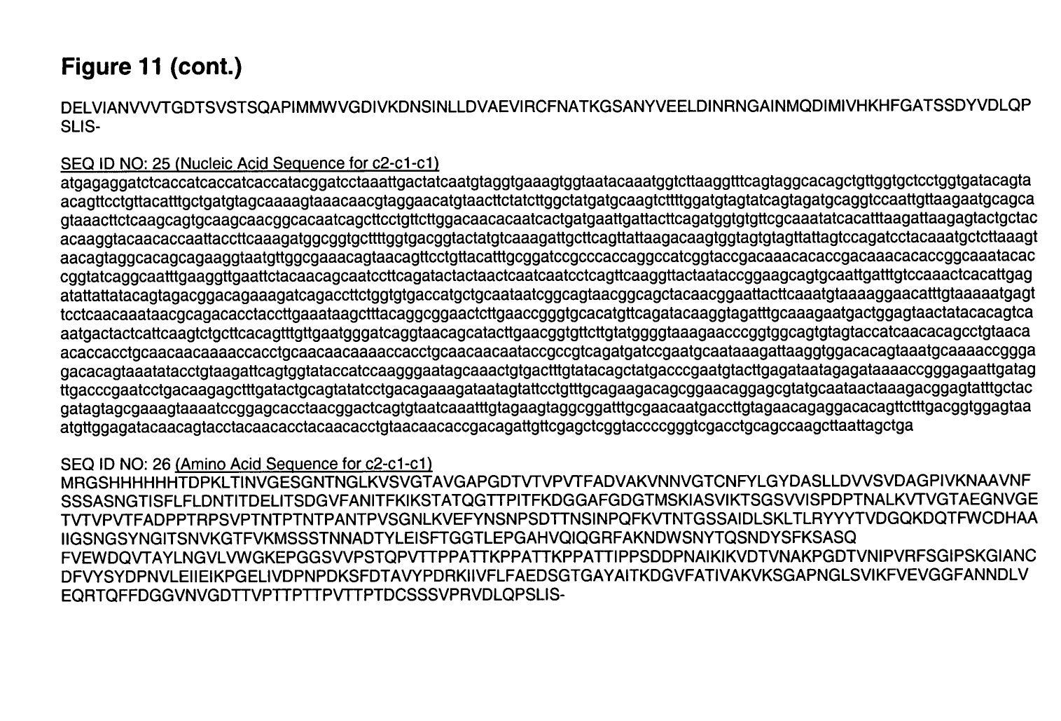

The pattern of products (glucose and cellodextrins) was identical from the free enzymes (SM901), the artificial complexes and the native cellulosomes for soluble as well as for insoluble cellulose (FIG. 11). In the case of insoluble cellulose as substrate, the main product is cellobiose with some glucose as a secondary product. The cellobiose has to be further degraded to glucose by addition of β-glucosidase to the complex. The β-glucosidase gene from Thermotoga neapolitana, genetically fused to a dockerin module, was used successfully and enhanced the production of reducing sugars about 2-fold.

Reconstitution of the cellulosome was thus possible. It could therefore be demonstrated for the first time that the order of components seems to be random along the scaffoldin and that the activity of the in vitro reconstituted cellulosome at least equalled that of the native cellulosome. The native cellulosome cannot be produced in industrial amounts.

### Results

The results showed clearly that the different cohesins bound the components equally and did not discriminate between cellulosomal components containing different dockerin modules. The binding to the cohesins was random. The assembly between cohesins and dockerins was fast and spontaneous. Once bound, the components were tightly fixed to the scaffoldin. An increase of the number of cohesins in the backbone scaffold by linking the individual backbone scaffold proteins by cohesin-dockerin interaction did increase the activity on crystalline cellulose (FIG. 6). Further, FIG. 6 shows that the addition of a certain type of CBM to the complex increased activity (FIG. 6). Furthermore, the “complete” scaffoldin forming a reconstituted cellulosome had the highest activity higher than that of the native cellulosome (even prior to systematic optimization).

### Example 4

### Optional Binding to Nano-Particles

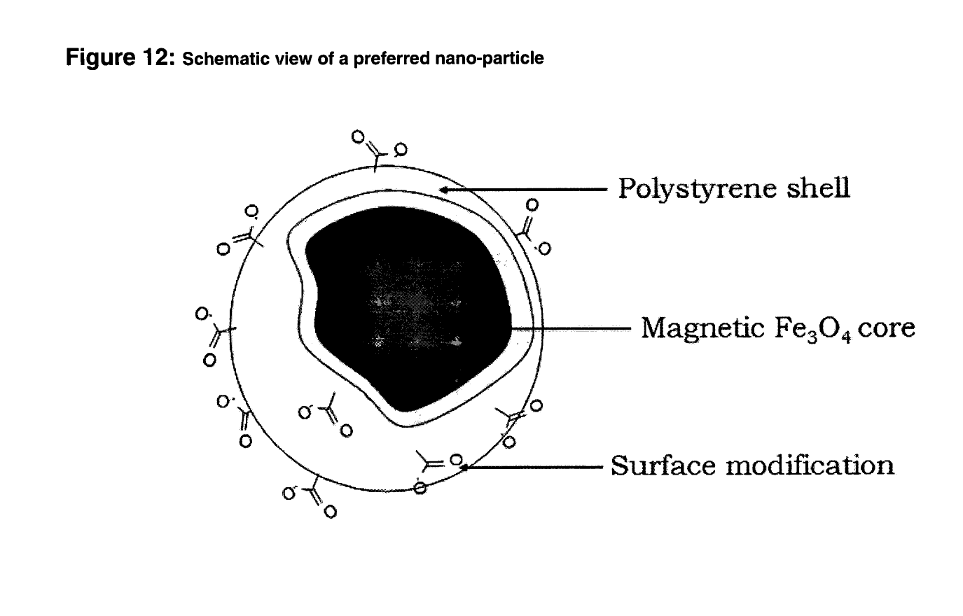

Nano-particles with an average diameter of 0.110±0.007 μm, a ferromagnetic core with superparamagnetic character and a polystyrene coating were chosen, which was chemically functionalized with COOH-residues on the surface. A heterobifunctional linker molecule was chemically coupled to the surface which binds the backbone scaffolds of choice; on the cohesions of the scaffold enzymes can be loaded by protein-protein interaction with the dockerins attached to the enzyme components (non-covalent cohesion-dockerin interaction). A schematic view of a preferred nano-particle is shown in FIG. 12.

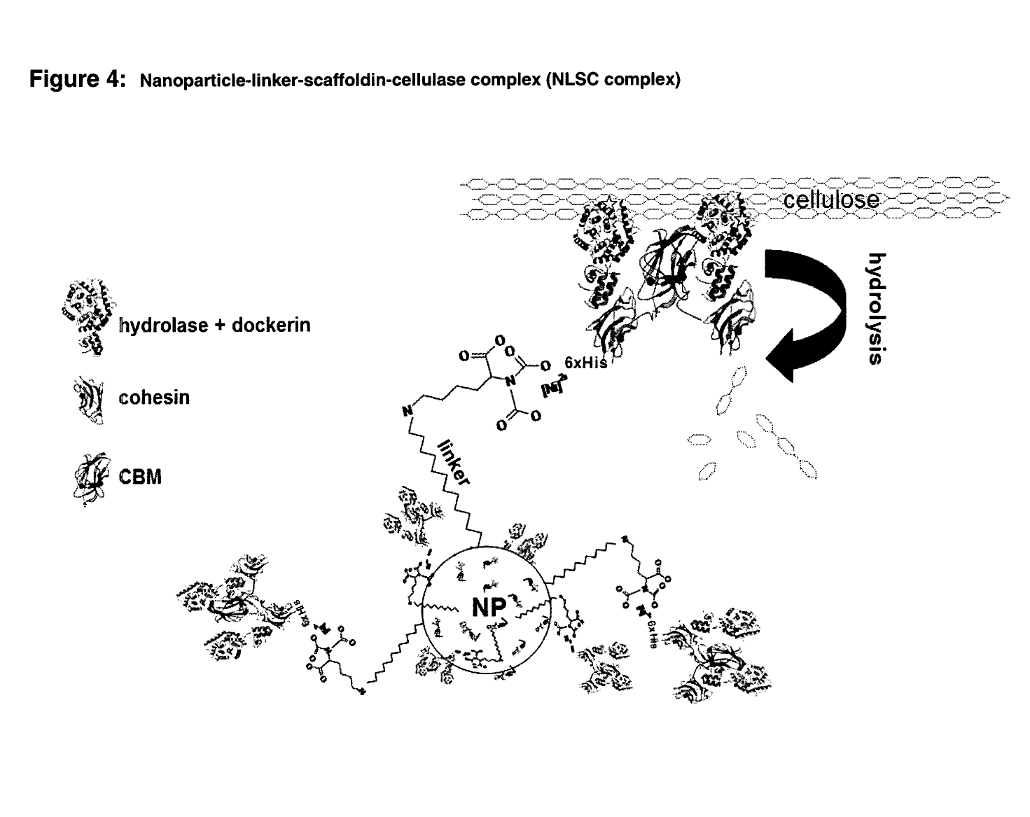

To bind the linker molecules to the surface of the nanoparticles, the functional (free COOH—) groups were activated. Water-soluble carbodiimide 1-ethyl-3-(3-dimethylaminopropyl) carbodiimide hydrochloride (EDC) forms active ester groups with carboxylate groups using the water-soluble compound N-hydroxysulfosuccinimide (sulfo-NHS). EDC reacts with a carboxylate group to form an active ester (O-acylisourea) leaving group. Sulfo-NHS esters are hydrophilic active groups that react rapidly with amines on target molecule¹⁸. However, in the presence of amine nucleophiles that can attack at the carbonyl group of the ester, the sulfo-NHS group rapidly leaves, creating a stable amide linkage with the amine. The advantage of adding sulfo-NHS to EDC is to increase the stability of the active intermediate, which ultimately reacts with the attacking amine. The reaction of EDC with carboxylate groups hydrolyses in aqueous solution within seconds. Forming a sulfo-NHS ester intermediate from the reaction of hydroxyl group on sulfo-NHS with the EDC active-ester complex extends the half-rate of the activated carboxylate to hours¹⁹. For the structure of the bound molecules on the surface of the nanoparticles see FIG. 4.

### A. Activation

20 mg of carboxyl-modified nanoparticles were washed three times in 2 ml activation buffer (50 mM MES, 0.5 M NaCl, pH 6.0) in a glass vial by separation with a strong NdFeB disc magnet (1.41-1.45 Tesla). The modified surface of the particles was activated by adding freshly prepared EDC solution and sulfo-NHS solution to a final concentration of 2 mM and 5 mM, respectively. The mixes reacted for 15 minutes at room temperature.

B. Coupling with Linker

Particles were washed two times with 2 ml reaction buffer (0.1 M sodium phosphate, 0.5 M NaCl, pH 7.2) by magnetical separation from the liquid. 5 mg of 0-(2-aminoethyl)-O-(2-carboxyethyl)-polyethylenglycol 3000 hydrochloride (NH2-PEG-COOH) was dissolved in 100 μl reaction buffer under nitrogen atmosphere and added to the activated particles. The covalent link between the activated particles and the amino groups of the PEG based linkers took place within 3 hours at RT. Buffer was changed to 2 ml activation buffer and the carboxylate group at the end of the covalently bound linker was activated with EDC and sulfo-NHS as described above. After two washing steps with 2 ml reaction buffer, 10 mg Nα,Nα-Bis(carboxymethyl)-L-lysine hydrate (NTA) was added. Coupling of NTA to the activated carboxylate groups of the linker took place within 3 hours at RT. Particles were washed three times with 2 ml distilled water. 1 ml of 1M NiSO₄ was added. Free Ni²⁺ ions were complexed by the carboxylate groups of NTA and NTA-Ni was formed. After 5 minutes the particles were washed two times with 2 ml distilled water and additionally two times with 2 ml 50 mM MOPS, 0.1 M NaCl, 5 mM CaCl₂, pH 6.0.

C. Conjugation of his-Tagged Protein Carriers

Backbone scaffold proteins were bound to the modified nanoparticles by incubating the particles described above with 1 to 1.5 mg protein overnight in 2 ml 50 mM MOPS, 0.1 M NaCl, 5 mM CaCl₂, pH 6.0.

Backbone scaffolds with different numbers of cohesins with and without a carbohydrate binding module (CBM) (CBM-c1-c1 and c1-c1) were immobilized on the surface modified nanoparticles.

The coupling efficiency of the proteins to the nano-particles was determined by spectrophotometric measurement of the optical absorption (590 nm, Bradford assay) of protein content before and after crosslinking. Protein loaded nanoparticles were magnetically separated from the reaction solution. The coupling efficiency was calculated by subtracting the remaining protein in the reaction solution from the initially applied amount of protein.

Reducing sugars were quantified at least in triplicate in the linear range of the reaction by the 3.5-dinitrosalicylic acid method²⁰, assuming that 1 unit of enzyme liberates 1 μmol of glucose equivalent per minute.

### Results

Surface binding (immobilization) has often been shown to stabilize enzymes and their activity. Experiments with cellulases have proved that direct immobilization of hydrolytic enzymes (cellulases from C. thermocellum) on the surface of nanoparticles diminished their specific activity. This indicates that active or structural domains of enzymes could be affected by direct covalent coupling to a surface. A directional specific coupling on cellulosomal backbone scaffolds was therefore chosen to maintain enzymatic activity.

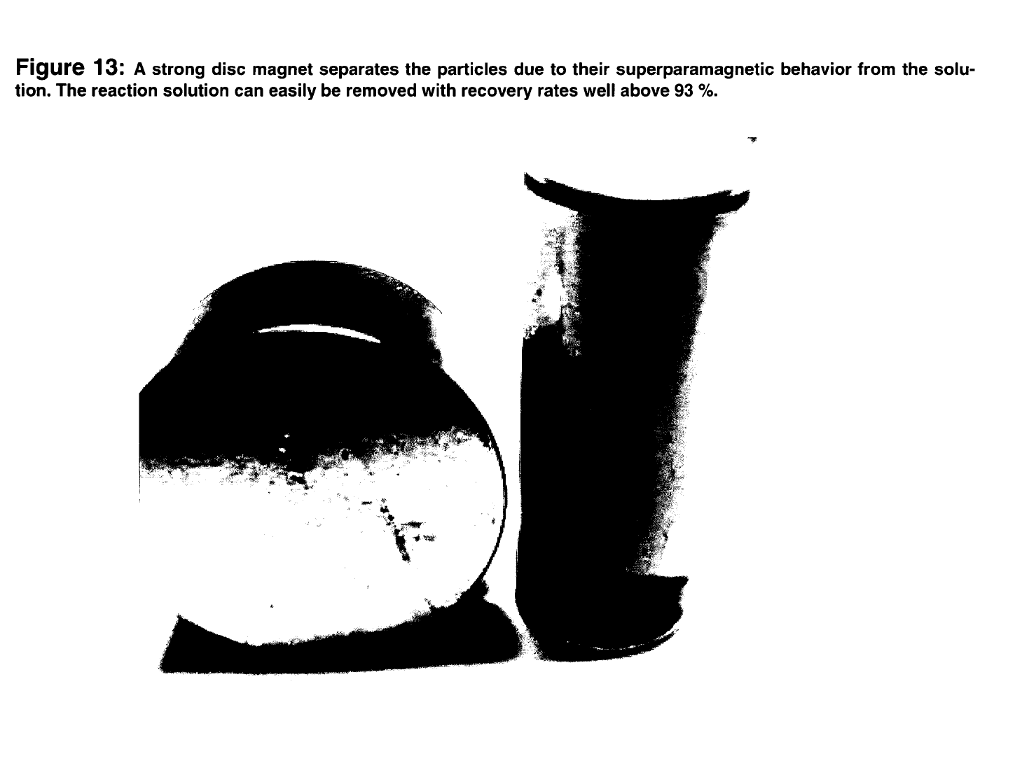

Best results of coupling of all reactions tested were obtained with 2 mmol EDC, 5 mmol Sulfo-NHS. A coupling efficiency of 80 μg/mg nanoparticles could be achieved. This corresponds to a calculated average number of ˜1300 backbone scaffold molecules per particle. No crosslinking between the carboxy modified particles was observed. Additionally, caused by the free carboxy groups and the resulting hydrophilic surface, COOH-beads resuspended very well. Separation is shown in FIG. 13.

For regeneration of the magnetite nanoparticles, particles were washed three times with EDTA (10 mM) to remove complexed Ni²⁺-ions and suspended in the same solution overnight at room temperature. After three washing steps with deionised water the particles were reloaded with Ni²⁺ and 6×His-tagged backbone scaffold proteins (calculated equimolar ratio of backbone scaffold proteins to enzyme components). 1 mg of fresh COOH modified nanoparticles could be loaded with ˜80 μg protein backbone scaffold protein. Recycled nanoparticles of the same amount were able to bind ˜50 μg (62.5% recovery) of scaffoldin proteins, and twice recycled nanoparticles could immobilize ˜25 μg (31.2% recovery) protein in this experiment.

Cellulases were immobilized by cohesin-dockerin recognition on the backbone scaffold-nanoparticle compound. The particles were loaded with SM901 mutant enzymes and their specific activity towards soluble, amorphous and insoluble cellulose was determined for production of reducing sugars with the dinitrosalicylic acid reagent as described elsewhere. The effectiveness of degradation depends on the type of substrate. Most accessible for hydrolysis is barley (3-glucan, a soluble β-1,3-1,4-glucan. The specific activity of the β-glucanases of the SM901 mutant enzymes towards barley (3-glucan was about 8 U/mg protein. The specific activity for degradation of carboxymethyl cellulose is about 1.1 U/mg protein. Amorphous cellulose (phosphoric acid swollen cellulose) is more accessible for degradation, resulting in specific activities of about 2.8 U/mg protein. A comparison with similar complexes without binding to nanoparticles shows that the immobilization of hydrolytic enzymes through cellulosomal-type backbone scaffolds had no negative effect on the degradation rate for all tested substrates.

On crystalline cellulose (MN300 or Avicel), free mutant enzymes showed a specific activity of 30 and 12 mU/mg respectively. Purified native cellulosomes with 9 cohesins and a carbohydrate binding module exhibited a specific activity of 423 mU/mg with MN300 as substrate, and 198 mU/mg with Avicel. The complexation of SM901 mutant enzymes with a backbone scaffold containing an increasing number of cohesions, the hydrolytic activity was enhanced to a specific activities of 63 and 28 mU/mg respectively. The enhancement of the activity was 2.1 and 2.3-fold over free enzymes. If a complex with three cohesins and a family-3 carbohydrate binding module was used, the degradation rate would increase 4.9 and 3.7-fold compared to unbound hydrolases (FIG. 5).

For comparison, backbone scaffold proteins without CBM or containing a CBM (c1-c1 and CBM-c1-c1) were used. The specific activity increased from 62 mU/mg to 102 mU/mg with MN300 as the substrate and 44 mU/mg to 108 mg/mU with Avicel if a CBM was present in the backbone scaffold.

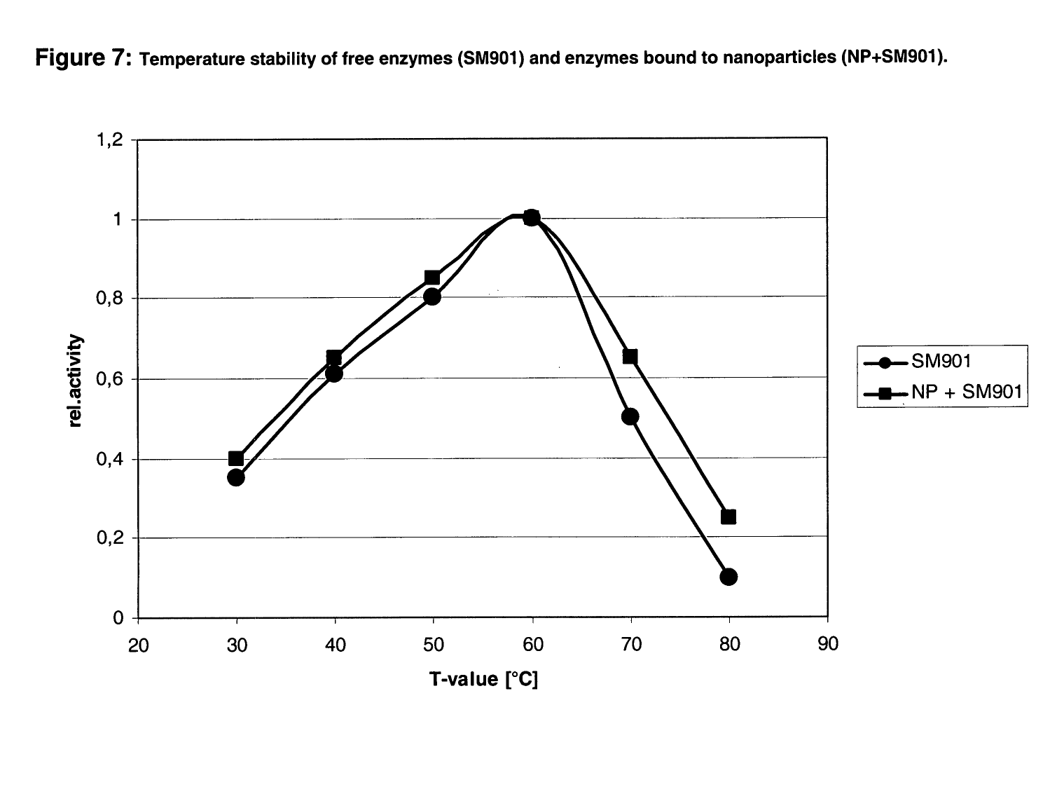

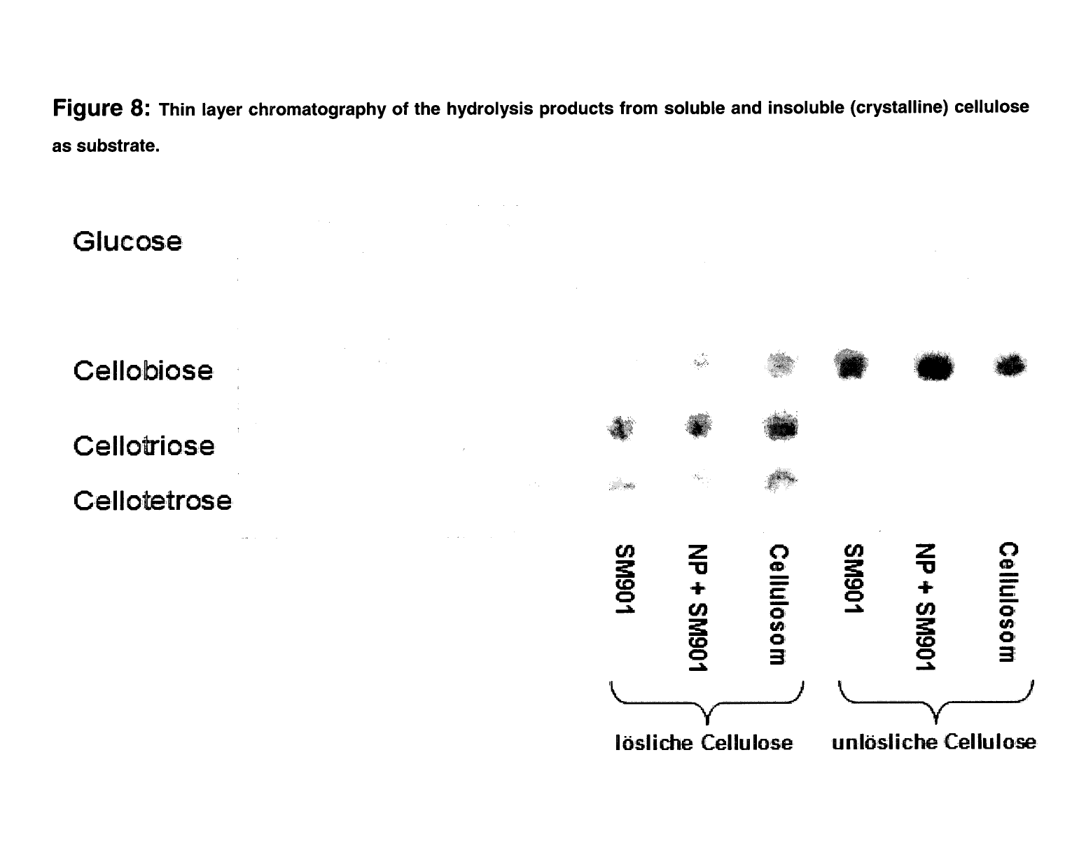

The immobilization of hydrolytic enzymes on nanoparticle-bound miniscaffoldins had no negative effect on the degradation rate of soluble and insoluble substrates. However, the pH stability and the temperature stability increased significantly (FIGS. 6 and 7 respectively).

### REFERENCES

¹ Schwarz, W. H., (2004). “Cellulose Struktur ohne Ende”, Naturwiss. Rundschau, 8:443-445.
² Singhania, R. R., et al. (2010), “Advancement and comparative profiles in the production technologies using solid-state and submerged fermentation for microbial cellulases”; Enz. Microb. Technol. 46:541-549
³ Lynd, L., Weimer, P. J., van Zyl, W. H., and Pretorius, I. S. (2002). Microbial cellulose utilization: fundamentals and biotechnology. Microbiol. Molec. Biol. Rev. 66, 506-577.
⁴ Shoham, Y., R. Lamed, and E. A. Bayer. 1999. The cellulosome concept as an efficient microbial strategy for the degradation of insoluble polysaccharides. Trends in Microbiol. 7:275-281.
⁵ Bayer, E. A., Y. Shoham, and R. Lamed. 2000. Cellulose-decomposing bacteria and their enzyme systems. In M. Dworkin, S. Falkow, E. Rosenberg, K.-H. Schleifer, and E. Stackebrandt (eds.), The Prokaryotes: An Evolving Electronic Resource for the Microbiological Community, 3rd edition. Springer-Verlag, New York.
⁶ Schwarz, W. H. 2001. The cellulosome and cellulose degradation by anaerobic bacteria. Appl. Microbiol. Biotechnol. 56:634-649.
Mechaly, A., S. Yaton, R. Lamed, H.-P. Fierobe, A. Belaich, J.-P. Belaich, Y. Shoham, and E. A. Bayer. 2000. Cohesin-dockerin recognition in cellulosome assembly: Experiment versus hypothesis. Proteins 39:170-177.
⁸ Leibovitz, E., H. Ohayon, P. Gounon, and P. Béguin. 1997. Characterization and subcellular localization of the Clostridium thermocellum scaffoldin dockerin binding protein SdbA. J. Bacteriol. 179:2519-2523.
⁹ Bayer, E. A., L. J. W. Shimon, Y. Shoham, and R. Lamed. 1998. Cellulosomes structure and ultrastructure. J. Struct. Biol. 124:221-234.
¹⁰ Zverlov, V. V., G. A. Velikodvorskaya, and W. H. Schwarz. 2003. Two new cellulosome components encoded downstream of cell in the genome of Clostridium thermocellum: the non-processive endoglucanase CelN and the possibly structural protein CseP. Microbiol. 149:515-524.
¹¹ Zverlov, V. V., Kellermann, J., & Schwarz, W. H. (2005). Functional subgenomics of Clostridium thermocellum cellulosomal genes: Identification of the major catalytic components in the extracellular complex and detection of three new enzymes. Proteomics, 5. 3646-3653.
¹² DOE Joint Genome Institute: http://genome.jgi-psf.org/microbial/
¹³ Gold N.D., & Martin V. J. (2007). Global view of the Clostridium thermocellum cellulosome revealed by quantitative proteomic analysis. J. Bacteriol. 189:6787-95.
¹⁴ Fierobe, H.-P. et al. (2002). Degradation of cellulose substrates by cellulosome chimeras. J. Biol. Chem. 277: 49621-49630.
¹⁵ Bayer, E. A., R. Kenig, & R. Lamed (1983). Adherence of Clostridium thermocellum to cellulose. J. Bacteriol. 156: 818-827.
¹⁶ Bayer, E. A., Y. Shoham, J. Tormo, & R. Lamed (1996). The cellulosome: a cell surface organelle for the adhesion to and degradation of cellulose. In: Bacterial adhesion: molecular and ecological diversity, pp. 155-182. Wiley-Liss, Inc.
¹⁷ Zverlov, V. V., Klupp, M., Krauss, J., & Schwarz, W. H. (2008). Mutants in the scaffoldin gene cipA of Clostridium thermocellum with impaired cellulosome formation and cellulose hydrolysis: insertions of a new IS-element, IS1447, and implications for cellulase synergism on crystalline cellulose. J. Bacteriol. 190: 4321-4327.
¹⁸ Staros, J. V., Wright, R. W., Swingle, D. M., (1986). Enhancement by N-hydroxysulfosuccinimide of water-soluble carbodiimide-mediated coupling reactions. Anal. Biochem., 156:220-222
¹⁹ Hermanson, G. T. (1995). Bioconjugate techniques. Academic Press
²⁰ Wood, T. M., and K. M. Bhat. 1988. Methods for measuring cellulase activities. Methods Enzymol. 160:87-112

1. A particle-free or particle-bound complex comprising:
a) a backbone scaffold comprising at least four binding sites, wherein at least two of the binding sites have essentially the same binding specificity; and
b) an enzyme component bound to each of said four binding sites, wherein at least three of said enzyme components are different enzyme components.

2. The complex of claim 1, wherein the complex is bound to a nano-particle.

3. The complex of claim 1, wherein the backbone scaffold is a linear, synthetic or biological backbone.

4. The complex of claim 1, wherein the backbone scaffold has at least four cohesin binding sites for dockerins.

5. The complex of claim 1, wherein the backbone scaffold comprises one or more proteins, wherein the one or more proteins are linked together by chemical interaction or by a cohesin-dockerin interaction, whereby the binding specificity of the linking interaction is different from the binding specificity of the enzymes.

6. The complex of claim 1, wherein the backbone scaffold is derived from a non-catalytic scaffolding protein from cellulolytic, cellulosome forming microorganisms or genetically modified derivatives thereof.

7. The complex of claim 1, wherein the backbone scaffold is derived from the non-catalytic scaffolding protein CipA from Clostridium thermocellum or genetically modified derivatives thereof.

8. The complex of claim 7, wherein the backbone scaffold comprises CBM-c1-c1-d3 as (SEQ ID NO: 24), c3-c1-c1-d2 (SEQ ID NO: 22), c2-c1-c1 (SEQ ID NO: 26), or derivatives thereof having more than 60% amino acid sequence identity in their cohesin modules.

9. The complex of claim 1, wherein the backbone scaffold comprises a carbohydrate binding module (CBM).

10. The complex of claim 9, wherein the carbohydrate binding module is a carbohydrate binding module (CBM3) from the cipA gene of Clostridium thermocellum that is integrated into or attached to the linear backbone scaffold.

11. The complex of claim 1, wherein the enzyme component comprises a dockerin module and a catalytic module of an enzyme.

12. The complex of claim 1, wherein the enzyme components are selected from the group consisting of: processive or non-processive endo-β-1,4-glucanases, processive exo-β-1,4-glucanases and glycosidases from polysaccharolytic microorganisms or genetically modified derivatives thereof.

13. The complex of claim 12, wherein the enzyme components are derived from dockerin module containing components of the Clostridium thermocellum cellulosome or from non-cellulosomal components of Clostridium thermocellum having a dockerin module fused thereto.

14. The complex of claim 1, wherein the enzyme components comprise CelK-d1 (SEQ ID NO: 8), CelR-d1 (SEQ ID NO: 10), CelT-d1 (SEQ ID NO: 14), CelE-d1 (SEQ ID NO: 16), CelS-d1 (SEQ ID NO: 6): and BglB-d1 (SEQ ID NO: 4) or derivatives thereof having more than 50% amino acid sequence identity in their dockerin modules.

15. A method for preparing the complex according to claim 1 comprising the steps:
a) recombinantly producing the enzyme components of claim 1,
b) recombinantly producing the backbone scaffold of claim 1,
c) mixing the purified, partially purified or non-purified components of a) and b) in vitro; and
d) randomly binding the enzyme components to the backbone scaffold.

16. The method of claim 15, further comprising the step of binding the recombinantly produced backbone scaffold or the recombinantly produced enzyme components to a particle

17. The method of claim 16, wherein the particle is a nano-particle.

18. The method of claim 15, wherein the total amount of backbone scaffolds in step c) and the total amount of enzyme components are mixed together in a molar ratio of 1 cohesin module to 1 enzyme component, and the at least three enzyme components are mixed together in a molar ratio of 1:1 to 1:15 to each other.

19. The complex produced by the method of claim 15.

20. A method for enzymatic hydrolysis of polysaccharide substrates comprising the steps of:
a) mixing the complex of claim 1 with insoluble cellulose; and
b) optionally isolating the degradation products.

21. (canceled)

22. The method of claim 20, wherein the polysaccharide substrate is crystalline cellulose or a crystalline cellulose containing substrate.

23. The complex of claim 2, wherein the nano-particle is a coated and chemically functionalized nano-particle.

24. The complex of claim 2, wherein the nano-particle is a poly-styrene coated ferromagnetic nanoparticle.

25. The method of claim 17, wherein the nano-particle is a poly-styrene coated ferromagnetic nano-particle.


SEQUENCE LISTING (26 sequences)

SEQ ID NO: 1 - DNA, 1488 bp, Clostridium thermocellum
  CelA-d1
atgagaggatcgcatcaccatcaccatcacggatccgcatgcgagctcaaaaaagtgaagaacgtaaaaaaaagagtaggtgtggttttgctgattcttgcagtgttgggggtttatatgttggcaatgccggcaaacactgtgtcagcggcaggtgtgccttttaacacaaaatacccctatggtcctacttctattgccgataatcagtcggaagtaactgcaatgctcaaagcagaatgggaagactggaagagcaagagaattacctcgaacggtgcaggaggatacaagagagtacagcgtgatgcttccaccaattatgatacggtatccgaaggtatgggatacggacttcttttggcggtttgctttaacgaacaggctttgtttgacgatttataccgttacgtaaaatctcatttcaatggaaacggacttatgcactggcacattgatgccaacaacaatgttacaagtcatgacggcggcgacggtgcggcaaccgatgctgatgaggatattgcacttgcgctcatatttgcggacaagttatggggttcttccggtgcaataaactacgggcaggaagcaaggacattgataaacaatctttacaaccattgtgtagagcatggatcctatgtattaaagcccggtgacagatggggaggttcatcagtaacaaacccgtcatattttgcgcctgcatggtacaaagtgtatgctcaatatacaggagacacaagatggaatcaagtggcggacaagtgttaccaaattgttgaagaagttaagaaatacaacaacggaaccggccttgttcctgactggtgtactgcaagcggaactccggcaagcggtcagagttacgactacaaatatgatgctacacgttacggctggagaactgccgtggactattcatggtttggtgaccagagagcaaaggcaaactgcgatatgctgaccaaattctttgccagagacggggcaaaaggaatcgttgacggatacacaattcaaggttcaaaaattagcaacaatcacaacgcatcatttataggacctgttgcggcagcaagtatgacaggttacgatttgaactttgcaaaggaactttatagggagactgttgctgtaaaggacagtgaatattacggatattacggaaacagcttgagactgctcactttgttgtacataacaggaaacttcccgaatcctttgagtgacctttccggccaaccgacaccaccgtcgaatccgacaccttcattgcctcctcaggttgtttacggtgatgtaaatggcgacggtaatgttaactccactgatttgactatgttaaaaagatatctgctgaagagtgttaccaatataaacagagaggctgcagacgttaatcgtgacggtgcgattaactcctctgacatgactatattaaagagatatctgataaagagcataccccacctaccttattag

SEQ ID NO: 2 - protein, 495 aa, Clostridium thermocellum
  CelA-d1
MRGSHHHHHHGSACELKKVKNVKKRVGVVLLILAVLGVYMLAMPANTVSAAGVPFNTKYPYGPTSIADNQSEVTAMLKAEWEDWKSKRITSNGAGGYKRVQRDASTNYDTVSEGMGYGLLLAVCFNEQALFDDLYRYVKSHFNGNGLMHWHIDANNNVTSHDGGDGAATDADEDIALALIFADKLWGSSGAINYGQEARTLINNLYNHCVEHGSYVLKPGDRWGGSSVTNPSYFAPAWYKVYAQYTGDTRWNQVADKCYQIVEEVKKYNNGTGLVPDWCTASGTPASGQSYDYKYDATRYGWRTAVDYSWFGDQRAKANCDMLTKFFARDGAKGIVDGYTIQGSKISNNHNASFIGPVAAASMTGYDLNFAKELYRETVAVKDSEYYGYYGNSLRLLTLLYITGNFPNPLSDLSGQPTPPSNPTPSLPPQVVYGDVNGDGNVNSTDLTMLKRYLLKSVTNINREAADVNRDGAINSSDMTILKRYLIKSIPHLPY

SEQ ID NO: 3 - DNA, 2502 bp, Thermotoga maritima
  BglB-d1
atgagaggatctcaccatcaccatcaccatgggatccaaatggcggtagatatcaagaaaataataaagcagatgactttggaagaaaaagcagggttgtgctcgggactggatttttggcataccaagcctgttgagagactgggcattccttcaataatgatgactgacggacctcatggactgagaaagcagagggaagatgcagagattgcggacatcaacaacagcgttccagcaacctgttttccgtctgcagcaggtttggcatgttcctgggacagagaactggttgagagagtaggtgcagcactaggagaagaatgtcaggcggaaaatgtctcaatactgcttggaccaggtgcaaatataaagcgttcacctttgtgtggaagaaattttgaatattttcccgaagacccttatctttcgtcagagctggcggcaagccatataaaaggagttcaaagtcagggagtgggtgcatgtcttaaacattttgccgcaaacaaccaggaacaccggagaatgaccgttgataccattgtagatgaaagaacgttgagggaaatatattttgcaagctttgagaatgctgtaaaaaaagcacggccttgggtggttatgtgtgcatataacaagctcaacggtgaatattgttcggagaacagatatcttttgacggaagttttaaagaatgaatggatgcatgacggctttgtggtatccgactggggtgcggtaaatgacagggtcagcggcctggatgcaggtcttgacctggaaatgcccaccagtcatggtattacggataaaaagatagttgaagccgtaaaaagcggaaagctgtctgaaaatattttaaacagagctgtggaaagaattttgaaagtaattattatggcactggaaaacaaaaaagaaaacgcgcagtatgaacaagatgctcatcacagactggcaaggcaggctgcggccgaatcgatggttcttcttaaaaacgaggacgatgtgcttcctttaaaaaagagcggaaccatagctttgataggagcttttgtgaaaaaaccaagataccagggttcgggcagttctcatattaccccgacaagacttgatgatatttatgaagagataaaaaaggccggagccgacaaagtaaaccttgtatattcggaaggatacaggcttgaaaatgacggtattgatgaggaattgataaacgaagctaaaaaggcggcatcaagctcggatgttgcggtagtatttgcagggcttccggatgaatatgaatctgaaggatttgacagaactcacatgagtattccggaaaatcaaaacaggctgatagaagcggtggccgaagtccagagtaatattgttgtggtattgcttaacggctcaccggttgaaatgccgtggattgacaaggtaaaatccgtgcttgaagcttatcttggaggccaggcgctgggaggccgctggcggatgtgctattcggtgaagtcaatcgtcggaaaacttgcggagaccttcccggtgaaattaagccataatccgtcctatttgaattttcccggagaggatgaccgagtggagtataaagaagggttgtttgtcggatacagatattatgatacaaagggaattgagccattgttcccctttggtcacggacttagctataccaaatttgaatacagtgatatatcagtcgataaaaaagatgtttcggacaatagcatcataaatgtcagcgttaaagtcaaaaatgttggaaaaatggcaggaaaagaaattgtgcagctgtatgtaaaagatgtgaaaagcagcgtcagaagacctgagaaagagcttaaaggatttgaaaaggtcttccttaatccgggagaagaaaagacggttacatttactttggacaaaagggcttttgcatattacaatactcagattaaggactggcatgttgaaagcggagagtttctgatattaataggaaggtcctccagggacatagttttaaaagaatcagtgagagtaaattcaacggtgaagataagaaaaagattcacagtgaattcagcggttgaagatgtaatgtccgattcttcggctgcggccgttttagggcctgtactaaaagagataaccgatgcactgcagattgatatggacaatgctcatgacatgatggcggccaatataaagaatatgcctttgcgctcacttgtcggttactctcagggaaggttaagcgaagaaatgctggaggaactggttgacaaaataaacaacgtcgactgcaatggcgacggaaaagttaattcaactgacgctgtggcattgaagagatatatcttgagatcaggtataagcatcaacactgataatgctgatgtaaatgctgatggcagagttaactctacagacttggcaatattgaagagatatattcttaaagagctcggtaccccgggtcgacctgcagccaagcttaattag

SEQ ID NO: 4 - protein, 833 aa, Thermotoga maritima
  BglB-d1
MRGSHHHHHHGIQMAVDIKKIIKQMTLEEKAGLCSGLDFWHTKPVERLGIPSIMMTDGPHGLRKQREDAEIADINNSVPATCFPSAAGLACSWDRELVERVGAALGEECQAENVSILLGPGANIKRSPLCGRNFEYFPEDPYLSSELAASHIKGVQSQGVGACLKHFAANNQEHRRMTVDTIVDERTLREIYFASFENAVKKARPWVVMCAYNKLNGEYCSENRYLLTEVLKNEWMHDGFVVSDWGAVNDRVSGLDAGLDLEMPTSHGITDKKIVEAVKSGKLSENILNRAVERILKVIIMALENKKENAQYEQDAHHRLARQAAAESMVLLKNEDDVLPLKKSGTIALIGAFVKKPRYQGSGSSHITPTRLDDIYEEIKKAGADKVNLVYSEGYRLENDGIDEELINEAKKAASSSDVAVVFAGLPDEYESEGFDRTHMSIPENQNRLIEAVAEVQSNIVVVLLNGSPVEMPWIDKVKSVLEAYLGGQALGGRWRMCYSVKSIVGKLAETFPVKLSHNPSYLNFPGEDDRVEYKEGLFVGYRYYDTKGIEPLFPFGHGLSYTKFEYSDISVDKKDVSDNSIINVSVKVKNVGKMAGKEIVQLYVKDVKSSVRRPEKELKGFEKVFLNPGEEKTVTFTLDKRAFAYYNTQIKDWHVESGEFLILIGRSSRDIVLKESVRVNSTVKIRKRFTVNSAVEDVMSDSSAAAVLGPVLKEITDALQIDMDNAHDMMAANIKNMPLRSLVGYSQGRLSEEMLEELVDKINNVDCNGDGKVNSTDAVALKRYILRSGISINTDNADVNADGRVNSTDLAILKRYILKELGTPGRPAAKLN

SEQ ID NO: 5 - DNA, 2274 bp, Clostridium thermocellum
  CelS-d1
atgagaggatctcaccatcaccatcaccatgggatccgcatgcagagaatggtaaaaagcagaaagatttctattctgttggcagttgcaatgctggtatccataatgatacccacaactgcattcgcaggtcctacaaaggcacctacaaaagatgggacatcttataaggatcttttccttgaactctacggaaaaattaaagatcctaagaacggatatttcagcccagacgagggaattccttatcactcaattgaaacattgatcgttgaagcgccggactacggtcacgttactaccagtgaggctttcagctattatgtatggcttgaagcaatgtatggaaatctcacaggcaactggtccggagtagaaacagcatggaaagttatggaggattggataattcctgacagcacagagcagccgggtatgtcttcttacaatccaaacagccctgccacatatgctgacgaatatgaggatccttcatactatccttcagagttgaagtttgataccgtaagagttggatccgaccctgtacacaacgaccttgtatccgcatacggtcctaacatgtacctcatgcactggttgatggacgttgacaactggtacggttttggtacaggaacacgggcaacattcataaacaccttccaaagaggtgaacaggaatccacatgggaaaccattcctcatccgtcaatagaagagttcaaatacggcggaccgaacggattccttgatttgtttacaaaggacagatcatatgcaaaacagtggcgttatacaaacgctcctgacgcagaaggccgtgctatacaggctgtttactgggcaaacaaatgggcaaaggagcagggtaaaggttctgccgttgcttccgttgtatccaaggctgcaaagatgggtgacttcttgagaaacgacatgttcgacaaatacttcatgaagatcggtgcacaggacaagactcctgctaccggttatgacagtgcacactaccttatggcctggtatactgcatggggtggtggaattggtgcatcctgggcatggaagatcggatgcagccacgcacacttcggatatcagaacccattccagggatgggtaagtgcaacacagagcgactttgctcctaaatcatccaacggtaagagagactggacaacaagctacaagagacagcttgaattctatcagtggttgcagtcggctgaaggtggtattgccggtggagcaaccaactcctggaacggtagatatgagaaatatcctgctggtacgtcaacgttctatggtatggcatatgttccgcatcctgtatacgctgacccgggtagtaaccagtggttcggattccaggcatggtcaatgcagcgtgtaatggagtactacctcgaaacaggagattcatcagttaagaatttgattaagaagtgggtcgactgggtaatgagcgaaattaagctctatgacgatggaacatttgcaattcctagcgacctcgagtggtcaggtcagcctgatacatggaccggaacatacacaggcaacccgaacctccatgtaagagtaacttcttacggtactgaccttggtgttgcaggttcacttgcaaatgctcttgcaacttatgccgcagctacagaaagatgggaaggaaaacttgatacaaaagcaagagacatggctgctgaactggttaaccgtgcatggtacaacttctactgctctgaaggaaaaggtgttgttactgaggaagcacgtgctgactacaaacgtttctttgagcaggaagtatacgttccggcaggttggagcggtactatgccgaacggtgacaagattcagcctggtattaagttcatagacatccgtacaaaatatagacaagatccttactacgatatagtatatcaggcatacttgagaggcgaagctcctgtattgaattatcaccgcttctggcatgaagttgaccttgcagttgcaatgggtgtattggctacatacttcccggatatgacatataaagtacctggtactccttctactaaattatacggcgacgtcaatgatgacggaaaagttaactcaactgacgctgtagcattgaagagatatgttttgagatcaggtataagcatcaacactgacaatgccgatttgaatgaagacggcagagttaattcaactgacttaggaattttgaagagatatattctcaaagaaatagatacattgccgtacaagaactaa

SEQ ID NO: 6 - protein, 757 aa, Clostridium thermocellum
  CelS-d1
MRGSHHHHHHGIRMQRMVKSRKISILLAVAMLVSIMIPTTAFAGPTKAPTKDGTSYKDLFLELYGKIKDPKNGYFSPDEGIPYHSIETLIVEAPDYGHVTTSEAFSYYVWLEAMYGNLTGNWSGVETAWKVMEDWIIPDSTEQPGMSSYNPNSPATYADEYEDPSYYPSELKFDTVRVGSDPVHNDLVSAYGPNMYLMHWLMDVDNWYGFGTGTRATFINTFQRGEQESTWETIPHPSIEEFKYGGPNGFLDLFTKDRSYAKQWRYTNAPDAEGRAIQAVYWANKWAKEQGKGSAVASVVSKAAKMGDFLRNDMFDKYFMKIGAQDKTPATGYDSAHYLMAWYTAWGGGIGASWAWKIGCSHAHFGYQNPFQGWVSATQSDFAPKSSNGKRDWTTSYKRQLEFYQWLQSAEGGIAGGATNSWNGRYEKYPAGTSTFYGMAYVPHPVYADPGSNQWFGFQAWSMQRVMEYYLETGDSSVKNLIKKWVDWVMSEIKLYDDGTFAIPSDLEWSGQPDTWTGTYTGNPNLHVRVTSYGTDLGVAGSLANALATYAAATERWEGKLDTKARDMAAELVNRAWYNFYCSEGKGVVTEEARADYKRFFEQEVYVPAGWSGTMPNGDKIQPGIKFIDIRTKYRQDPYYDIVYQAYLRGEAPVLNYHRFWHEVDLAVAMGVLATYFPDMTYKVPGTPSTKLYGDVNDDGKVNSTDAVALKRYVLRSGISINTDNADLNEDGRVNSTDLGILKRYILKEIDTLPYKN

SEQ ID NO: 7 - DNA, 2655 bp, Clostridium thermocellum
  CelK-d1
atgagaggatctcaccatcaccatcaccatgggatccgcatgcgagctctggaagacaagtcttcaaagttgccagattataaaaacgaccttttgtatgaaagaacattcgacgaaggtctttgctttccgtggcatacttgcgaagacagtggaggaaaatgtgatttcgctgttgttgatgttccaggagagcctgggaacaaagctttccgcttgacagtaattgacaaaggacaaaacaagtggagtgtccagatgagacacagaggtattaccctcgagcaaggacatacatacacggtaaggtttacgatttggtctgacaaatcctgtagggtttatgctaaaattggtcagatgggtgaaccctatactgaatattggaacaataactggaatccattcaaccttacaccaggacagaagcttacagttgaacagaattttacaatgaactatcctactgatgacacatgcgagttcacattccatttgggtggagaacttgctgcaggtacaccttactatgtttaccttgatgatgtatctctctacgatcctaggtttgtaaagcctgttgaatatgtacttccgcagccggatgtacgtgttaaccaggtaggatacttgccgtttgcaaagaagtatgctactgttgtatcttcttcaaccagcccgcttaagtggcagcttctcaattcggcaaatcaggttgttttggaaggtaatacaataccaaaaggacttgacaaagattcacaggattatgtacattggatagatttctccaactttaagactgaaggaaaaggttattacttcaagcttccgactgtaaacagcgatacaaattacagccatcctttcgatatcagtgctgatatttactccaagatgaaatttgatgcattggcattcttctatcacaagagaagcggtattcctattgaaatgccgtatgcaggaggagaacagtggaccagacctgcaggacatattggaattgagccgaacaagggagatacaaatgttcctacatggcctcaggatgatgaatatgcaggaagacctcaaaaatattatacaaaagatgtaaccggtggatggtatgatgccggtgaccacggtaaatatgttgtaaacggcggtatagctgtttggacattgatgaacatgtatgaaagggcaaaaatcagaggcatagctaatcaaggtgcttataaagacggtggaatgaacataccggagagaaataacggttatccggacattcttgatgaagcaagatgggaaattgagttctttaagaaaatgcaggtaactgaaaaagaggatccttccatagccggaatggtacaccacaaaattcacgacttcagatggactgctttgggtatgttgcctcacgaagatccccagccacgttacttaaggccggtaagtacggctgcgactttgaactttgcggcaactttggcacaaagtgcacgtctttggaaagattatgatccgacttttgctgctgactgtttggaaaaggctgaaatagcatggcaggcggcattaaagcatcctgatatttatgctgagtatactcccggtagcggtggtcccggaggcggaccatacaatgacgactatgtcggagacgaattctactgggcagcctgcgaactttatgtaacaacaggaaaagacgaatataagaattacctgatgaattcacctcactatcttgaaatgcctgcaaagatgggtgaaaacggtggagcaaacggagaagacaacggattgtggggatgcttcacctggggaactactcaaggattgggaaccattactcttgcattggttgaaaacggattgcctgctacagacattcaaaaggcaagaaacaatatagctaaagctgctgacagatggcttgagaatattgaagagcaaggttacagactgccgatcaaacaggcggaggatgagagaggcggttatccatggggttcaaactccttcattttgaaccagatgatagttatgggatacgcatatgactttacaggcaacagcaagtatcttgacggaatgcaggatggtatgagctacctgttgggaagaaacggactggatcagtcctatgtaacagggtatggtgagcgtccacttcagaatcctcatgacagattctggacgccgcagacaagtaagaaattccctgctccacctccgggtataattgccggtggtccgaactcccgtttcgaagacccgacaataactgcagcagttaagaaggatacaccgccgcagaagtgctacattgaccatacagactcatggtcaaccaacgagataactgttaactggaatgctccgtttgcatgggttacagcttatctcgatgaaattgacttaataacaccgccaggaggagtagacccagaagaaccggaggttatttatggtgactgcaatggcgacggaaaagttaattcaactgacgctgtggcattgaagagatatatcttgagatcaggtataagcatcaacactgataatgctgatgtaaatgctgatggcagagttaactctacagacttggcaatattgaagagatatattcttaaagagatagatgtattgccacataaataa

SEQ ID NO: 8 - protein, 884 aa, Clostridium thermocellum
  CelK-d1
MRGSHHHHHHGIRMRALEDKSSKLPDYKNDLLYERTFDEGLCFPWHTCEDSGGKCDFAVVDVPGEPGNKAFRLTVIDKGQNKWSVQMRHRGITLEQGHTYTVRFTIWSDKSCRVYAKIGQMGEPYTEYWNNNWNPFNLTPGQKLTVEQNFTMNYPTDDTCEFTFHLGGELAAGTPYYVYLDDVSLYDPRFVKPVEYVLPQPDVRVNQVGYLPFAKKYATVVSSSTSPLKWQLLNSANQVVLEGNTIPKGLDKDSQDYVHWIDFSNFKTEGKGYYFKLPTVNSDTNYSHPFDISADIYSKMKFDALAFFYHKRSGIPIEMPYAGGEQWTRPAGHIGIEPNKGDTNVPTWPQDDEYAGRPQKYYTKDVTGGWYDAGDHGKYVVNGGIAVWTLMNMYERAKIRGIANQGAYKDGGMNIPERNNGYPDILDEARWEIEFFKKMQVTEKEDPSIAGMVHHKIHDFRWTALGMLPHEDPQPRYLRPVSTAATLNFAATLAQSARLWKDYDPTFAADCLEKAEIAWQAALKHPDIYAEYTPGSGGPGGGPYNDDYVGDEFYWAACELYVTTGKDEYKNYLMNSPHYLEMPAKMGENGGANGEDNGLWGCFTWGTTQGLGTITLALVENGLPATDIQKARNNIAKAADRWLENIEEQGYRLPIKQAEDERGGYPWGSNSFILNQMIVMGYAYDFTGNSKYLDGMQDGMSYLLGRNGLDQSYVTGYGERPLQNPHDRFWTPQTSKKFPAPPPGIIAGGPNSRFEDPTITAAVKKDTPPQKCYIDHTDSWSTNEITVNWNAPFAWVTAYLDEIDLITPPGGVDPEEPEVIYGDCNGDGKVNSTDAVALKRYILRSGISINTDNADVNADGRVNSTDLAILKRYILKEIDVLPHK

SEQ ID NO: 9 - DNA, 2181 bp, Clostridium thermocellum
  CelR-d1
atgagaggatctcaccatcaccatcaccatacggatcctgtttttgcagcagactataactatggagaagcactccaaaaagcaattatgttctatgaatttcaaatgtccggaaagcttcccgacaacatccgtaacaactggcgcggtgattcatgtctcggagacggaagcgatgtaggtcttgacctcacaggaggttggtttgacgccggtgaccatgtaaaattcaatctgcctatggcttacacagccactatgcttgcatgggctgtgtatgagtacaaggacgcgttacaaaaaagcggtcaattgggctatttaatggatcagattaaatgggcatcggactacttcataagatgccatcccgaaaaatatgtatattattatcaagtgggtaacggtgacatggaccacagatggtgggtgccggcagaatgtatagatgttcaggcaccaagaccgtcttacaaagtagatctgtcaaatcccggttccacagttactgcgggtacagctgccgcacttgctgcaactgccttggtattcaaaggcactgatccggcatatgccgctctgtgcatacgtcatgcagaagaactctttgattttgctgaaaccactatgagtgataaaggatataccgcagcattgaatttctacacatctcacagtggatggtatgacgagctttcctgggcaggtgcatggatttatcttgcagacggtgacgaaacttatcttgaaaaagctgaaaagtatgtggataaatggccaatcgaaagccagacaacttacattgcttattcatggggtcactgctgggacgacgttcactacggagcagcacttcttttggcaaagattacaaacaaatccttatacaaagaagcgatagaaagacacctggactattggacagttggatttaatggtcagagagtcagatatacaccaaagggtcttgctcacctcactgactggggtgtattaagacatgccactactactgcattccttgcatgtgtttattccgactggtcagaatgtccaagggaaaaagccaatatttacatagattttgccaagaaacaggctgactatgccttaggcagcagcggcagaagttatgtagtcggatttggtgtaaatcctccgcagcatccgcaccacagaactgcccacagctcatggtgtgacagtcaaaaagttcctgaataccacagacacgttctttacggagcactcgtaggcggacctgatgccagcgatgcttatgttgatgatataggaaactatgtaacaaatgaggttgcctgcgactacaatgccggttttgtaggattgctcgccaagatgtatgaaaaatatggcggaaaccccataccaaacttcatggctatagaagaaaaaacaaatgaagaaatttatgttgaagctaccgccaattcaaataacggtgtcgaattgaaaacatacctttacaataaatccggatggccggcaagagtttgcgacaagctttccttcagatatttcatggaccttacggaatatgtatccgccggatacaatcctaatgatataactgtttctataatttacagtgcagcaccaactgcaaaaatttcaaaaccaatactttatgacgcatccaaaaacatatattattgcgaaatcgatctctccggtaccaagatattccccggaagcaactcagaccaccagaaagaaacccaatttagaatacagcctcctgcaggcgcaccttgggacaacaccaacgacttctcctatcagggaatcaagaaaaacggtgaagttgtaaaagaaatgcctgtttatgaagacggagttctcatattcggtgtagaacccaatggtaccggtcctgcaacaccaacgccgaaaccgtccgtaaatccttcaccttcacctacgccaacatcggatattctttacggtgacatcaatctggacggaaaaattaactcttcagatgttacactgttaaaaagatatattgtgaagtccatagatgttttcccaaccgctgatccggaacggagcttaatagcatcagatgtaaacggagacggaagggtaaactctacagactattcataccttaaacgttatgtcttgaaaatcataccaaccatacccggaaattcatga

SEQ ID NO: 10 - protein, 726 aa, Clostridium thermocellum
  CelR-d1
MRGSHHHHHHTDPVFAADYNYGEALQKAIMFYEFQMSGKLPDNIRNNWRGDSCLGDGSDVGLDLTGGWFDAGDHVKFNLPMAYTATMLAWAVYEYKDALQKSGQLGYLMDQIKWASDYFIRCHPEKYVYYYQVGNGDMDHRWWVPAECIDVQAPRPSYKVDLSNPGSTVTAGTAAALAATALVFKGTDPAYAALCIRHAEELFDFAETTMSDKGYTAALNFYTSHSGWYDELSWAGAWIYLADGDETYLEKAEKYVDKWPIESQTTYIAYSWGHCWDDVHYGAALLLAKITNKSLYKEAIERHLDYWTVGFNGQRVRYTPKGLAHLTDWGVLRHATTTAFLACVYSDWSECPREKANIYIDFAKKQADYALGSSGRSYVVGFGVNPPQHPHHRTAHSSWCDSQKVPEYHRHVLYGALVGGPDASDAYVDDIGNYVTNEVACDYNAGFVGLLAKMYEKYGGNPIPNFMAIEEKTNEEIYVEATANSNNGVELKTYLYNKSGWPARVCDKLSFRYFMDLTEYVSAGYNPNDITVSIIYSAAPTAKISKPILYDASKNIYYCEIDLSGTKIFPGSNSDHQKETQFRIQPPAGAPWDNTNDFSYQGIKKNGEVVKEMPVYEDGVLIFGVEPNGTGPATPTPKPSVNPSPSPTPTSDILYGDINLDGKINSSDVTLLKRYIVKSIDVFPTADPERSLIASDVNGDGRVNSTDYSYLKRYVLKIIPTIPGNS

SEQ ID NO: 11 - DNA, 4845 bp, Clostridium thermocellum
  CelJ-d1
atgagaggatctcaccatcaccatcaccatgggatccgcatgccaaagagaagattatcgctacttttggtacttgccataatgtttacgatggtcgttccacagatatctgcaagtgccgaaacagttgctcctgaaggctacaggaagcttttggatgtacaaattttcaaggattcgcctgtagtcggatggtcaggaagcggtatgggcgagcttgaaactatcggcgatacccttccggttgataccacagttacatataacggtttgccgactttaagactgaatgtccagacaaccgttcagtcaggatggtggatttctcttcttacattaagaggatggaacacccatgacctttcccagtatgtcgaaaacggttatcttgagtttgacatcaagggtaaggaaggcggagaagactttgttattggtttcagggacaaggtttatgaacgcgtttacggacttgaaattgatgttaccacagtaatatcaaattatgtaacggtaactacggactggcagcatgttaagattcctttgagagacctgatgaagattaataacggatttgatccttcatcagttacatgcctggtgttctcaaaaagatatgcagatccgtttacagtatggttcagtgatataaagattacatcagaagacaatgaaaagtccgctcctgcaatcaaggtaaaccagcttggctttattcctgaagctgaaaaatacgctttggttacaggttttgcagaagagctcgcagtatcggaaggtgacgaatttgccgttataaatgctgcggacaattctgttgcttataccggaaaattaactcttgtaacagaatatgaacctcttgattccggagaaaaaatacttaaggcagatttcagcgacttgactgtacctggcaaatactacattagtattgaaggtcttgacaattcacccaagtttgaaatcggtgaaggtatttacggtccactggttgttgacgctgcaagatatttctattatcagcgtcagggtatagaacttgaagagccttatgcgcagggatatccccgcaaggacgttactcctcaggacgcatatgctgtatttgcatccggaaagaaggatccgattgacataacaaagggttggtatgacgcaggagacttcggtaagtatgtaaatgccggagcaaccggtgtttccgatttgttctgggcatatgaaatgttcccttcccagtttgttgacggtcagttcaatattcctgaaagcggaaacggtgtaccggacatccttgacgaagctcgctgggagcttgaatggatgctgaaaatgcaggacaaagaaagcggaggattctatcccagagttcaatctgacaatgacgaaaacataaaatcaagaataatcagggatcagaacggctgtaccactgatgatactgcatgtgccgccggaatacttgctcatgcatacttgatttacaaggatattgaccctgattttgcacaagagtgcctggatgcggcaataaatgcatggaaattccttgaaaagaatcctgaaaacattgtttcacctccgggtccatacaacgtatatgacgacagcggagacagactctgggctgcagcttcgctgtacagagctaccggtgaagaggtttatcatacatactttaaacaaaactacaaatcttttgcacaaaagttcgaaagcccgactgcatatgctcatacatggggtgatatgtggcttacggcattcctttcgtatttgaaagctgaaaacaaggatcaggaagttgtagactggattgatacagagtttggaatctggcttgaaaacatactcacaagatatgagaacaatccatggaagaatgcaattgttcccggaaactacttctggggaatcaacatgcaggttatgaatgttccgatggatgctatcataggttcacagcttcttggaaaatacagtgacagaatagaaaaattaggttttggttcacttaactggctgcttggtacaaatccgcttcgcttcagctttgtatcaggatatggagaggattctgtaaaaggagtattcagcaatatttacaatacggacggcaagcagggaattccgaaaggatacatgcctggtggaccaaatgcttatgaaggtgcaggcctgtcaaggtttgcagcaaaatgctacaccagaagtaccggtgactgggtagccaacgaacatacagtatattggaactcagctttggtatttatggctgcttttgcaaaccagggttcagaggttaatccgggacctgcgccggaaccgggagtaactccgaatcctacagaacctgcaaaagtggttgacatcaggatagatacttctgctgaaagaaagccaatcagcccgtatatatacggaagcaatcaggaacttgatgcaacagttactgcaaagaggttcggcggaaacagaactacaggatacaactgggaaaacaacttctcaaatgcaggaagtgactggctgcattacagtgatacataccttttggaggacggcggagttcctaagggagagtggagtacacctgcttctgtagttaccacgttccatgacaaggcacttagcaaaaatgttccttacacacttatcactcttcaggcagcaggttatgtttccgcagacggaaacggaccggtttcccaggaagaaactgcaccgtcttcaagatggaaggaagttaagtttgaaaagggagcacctttctcacttacaccggacacagaagatgattatgtttacatggatgagtttgtaaactatcttgtaaacaaatacggaaatgcatccacacctacaggaataaagggttattcaatagataacgagccggcattgtggagtcatactcatccgagaattcatccggacaatgtaactgccaaagagcttattgaaaaatctgtagctctttccaaggcggttaaaaaggtagatccatatgcagaaatattcggacctgctttgtacggatttgccgcatatgagacacttcagtcagctcctgactggggaactgaaggagaaggatacaggtggtttatagattattacctcgataagatgaaaaaggcttctgatgaagaaggaaagagacttttggacgtacttgacgtacactggtatccggaagccaggggcggcggtgaaagaatatgctttggagccgatccaagaaatattgagacaaacaaagcaagattgcaggcgcccagaacattgtgggatcctacatatattgaagacagctggataggacaatggaagaaggatttcctcccgatattacctaatcttttggattccattgaaaaatattatccgggaacgaagcttgctataactgaatatgactatggcggaggaaatcatattacaggcggtattgctcaagccgatgttcttggtatattcggtaaatacggtgtttaccttgcaacattctggggagatgcaagcaataactatactgaggccggtataaacctttataccaactacgacggcaaaggcggcaaatttggagatacatccgtaaaatgtgaaacgtccgacatagaagtaagctctgcttatgcatccattgtcggtgaagatgacagcaaactccatatcattcttttgaacaagaactatgaccagccgacgacattcaatttctcaattgacagcagcaagaactacacaataggaaatgtatgggcatttgacagaggaagctccaatattactcaaagaactcctatagtgaacataaaggacaataccttcacatatacagtaccggctttgacagcgtgccatattgtgcttgaagctgcggagcccgtagtgtacggagacttgaacaatgactctaaagtaaacgcagtagacattatgatgctcaaacgatatattctcggaataatagataatataaatctgacagcagctgacatttattttgacggtgttgtaaattcaagtgactataatataatgaagagatatttgttaaaggcaatagaagatattccttatgttccggaaaaccaggcacctaaagcaatatttactttctcgcccgaagacccggttactgacgagaatgtagtgttcaatgcatcaaattcaatagatgaagacggaacaattgcctattatgcatgggatttcggtgacggatatgaaggaacttcaacaacaccgactattacctataagtataaaaaccccggaacatacaaagtaaaactgattgttacagacaaccagggggcttcaagttcgtttacagctaccataaaagtaacctcagctaccggggacaattccaaattcaactttgaagacggcacgctgggaggatttacaacatccggaacaaatgctacgggtgttgttgtgaacactactgaaaaagcattcaaaggcgaaagaggtcttaaatggactgtaacaagcgaaggagaaggaactgcagaattgaaacttgacggaggtactattgtagttcccggtaccactatgacgtttagaatctggataccttccggtgcgcctattgctgccatccagccgtatattatgcctcatacacctgattggtcggaagtcctctggaattcgacatggaaaggatacaccatggtgaagaccgatgactggaatgaaattaccctgacactgccggaagacgtggatccgacttggccgcagcagatgggtatacaggtacagaccatagatgaaggtgaattcactatctatgtagatgctattgactggtaa

SEQ ID NO: 12 - protein, 1614 aa, Clostridium thermocellum
  CelJ-d1
MRGSHHHHHHGIRMPKRRLSLLLVLAIMFTMVVPQISASAETVAPEGYRKLLDVQIFKDSPVVGWSGSGMGELETIGDTLPVDTTVTYNGLPTLRLNVQTTVQSGWWISLLTLRGWNTHDLSQYVENGYLEFDIKGKEGGEDFVIGFRDKVYERVYGLEIDVTTVISNYVTVTTDWQHVKIPLRDLMKINNGFDPSSVTCLVFSKRYADPFTVWFSDIKITSEDNEKSAPAIKVNQLGFIPEAEKYALVTGFAEELAVSEGDEFAVINAADNSVAYTGKLTLVTEYEPLDSGEKILKADFSDLTVPGKYYISIEGLDNSPKFEIGEGIYGPLVVDAARYFYYQRQGIELEEPYAQGYPRKDVTPQDAYAVFASGKKDPIDITKGWYDAGDFGKYVNAGATGVSDLFWAYEMFPSQFVDGQFNIPESGNGVPDILDEARWELEWMLKMQDKESGGFYPRVQSDNDENIKSRIIRDQNGCTTDDTACAAGILAHAYLIYKDIDPDFAQECLDAAINAWKFLEKNPENIVSPPGPYNVYDDSGDRLWAAASLYRATGEEVYHTYFKQNYKSFAQKFESPTAYAHTWGDMWLTAFLSYLKAENKDQEVVDWIDTEFGIWLENILTRYENNPWKNAIVPGNYFWGINMQVMNVPMDAIIGSQLLGKYSDRIEKLGFGSLNWLLGTNPLRFSFVSGYGEDSVKGVFSNIYNTDGKQGIPKGYMPGGPNAYEGAGLSRFAAKCYTRSTGDWVANEHTVYWNSALVFMAAFANQGSEVNPGPAPEPGVTPNPTEPAKVVDIRIDTSAERKPISPYIYGSNQELDATVTAKRFGGNRTTGYNWENNFSNAGSDWLHYSDTYLLEDGGVPKGEWSTPASVVTTFHDKALSKNVPYTLITLQAAGYVSADGNGPVSQEETAPSSRWKEVKFEKGAPFSLTPDTEDDYVYMDEFVNYLVNKYGNASTPTGIKGYSIDNEPALWSHTHPRIHPDNVTAKELIEKSVALSKAVKKVDPYAEIFGPALYGFAAYETLQSAPDWGTEGEGYRWFIDYYLDKMKKASDEEGKRLLDVLDVHWYPEARGGGERICFGADPRNIETNKARLQAPRTLWDPTYIEDSWIGQWKKDFLPILPNLLDSIEKYYPGTKLAITEYDYGGGNHITGGIAQADVLGIFGKYGVYLATFWGDASNNYTEAGINLYTNYDGKGGKFGDTSVKCETSDIEVSSAYASIVGEDDSKLHIILLNKNYDQPTTFNFSIDSSKNYTIGNVWAFDRGSSNITQRTPIVNIKDNTFTYTVPALTACHIVLEAAEPVVYGDLNNDSKVNAVDIMMLKRYILGIIDNINLTAADIYFDGVVNSSDYNIMKRYLLKAIEDIPYVPENQAPKAIFTFSPEDPVTDENVVFNASNSIDEDGTIAYYAWDFGDGYEGTSTTPTITYKYKNPGTYKVKLIVTDNQGASSSFTATIKVTSATGDNSKFNFEDGTLGGFTTSGTNATGVVVNTTEKAFKGERGLKWTVTSEGEGTAELKLDGGTIVVPGTTMTFRIWIPSGAPIAAIQPYIMPHTPDWSEVLWNSTWKGYTMVKTDDWNEITLTLPEDVDPTWPQQMGIQVQTIDEGEFTIYVDAIDW

SEQ ID NO: 13 - DNA, 1809 bp, Clostridium thermocellum
  CelT-d1
atgagaggatctcaccatcaccatcaccatacggatccagggattgtctctttcaacaccgtaagcaccagtgccgccggagaatacaattatgcaaaggcgctgcagtattccatgttcttctatgatgcgaacatgtgcggtacaggtgttgacgagaacagccttttgtcatggagaggagactgccacgtatatgatgcaagacttcctctggattcccagaacaccaacatgtccgatggttttataagcagcaacagaagtgtgcttgaccctgacggagacggcaaagttgacgtgtcaggcggttttcatgacgccggcgaccatgtgaagtttggtttgcctgaggcttatgccgcttcaacagtgggttggggttactatgaatttaaagaccagttccgtgcaacgggacaggccgtccatgctgaagtaattttaagatacttcaatgactattttatgagatgtactttcagagacgcttccggaaatgttgtggcgttctgtcatcaggtgggcgacggagatatcgaccatgcattttggggtgctccggaaaatgacaccatgttcagaagaggttggtttattaccaaagaaaagcctggaactgacattatttcggcaacagcagcttctttagcaataaactacatgaattttaaagacacagaccctcaatatgcggcaaaaagccttgattatgcaaaagctttgtttgattttgcggagaaaaatccaaaaggggtagttcagggagaggacggaccaaaaggttattatggttcaagcaaatggcaggatgactactgctgggctgccgcatggctttatttggcaacgcagaatgagcactatttggatgaagcatttaaatattatgattattatgctccgccgggatggatacattgctggaatgacgtgtggtcgggaaccgcatgtattttggcggaaataaatgatttgtacgacaaggacagccagaatttcgaagacaggtataaaagagcttccaataagaatcagtgggagcagatagacttctggaaacccatacaagatttgcttgacaagtggtcgggtggcggtattacagttacaccgggcggatacgttttcctcaatcagtggggttctgcaagatacaatactgccgctcagctgatagctcttgtttatgacaagcatcatggtgacacaccgtcaaaatatgctaactgggcacggtcgcagatggattatctgttgggtaaaaacccgttgaatcgctgctatgttgtaggctacagcagcaattcggtcaaatacccgcaccacagagcggcttccggactgaaagatgccaatgattcttctccgcacaaatatgtgttgtatggtgccctggtcggagggccggatgcaagtgaccagcatgtggatagaacaaatgattatatttacaatgaggttgccattgactataatgccgcttttgtgggagcatgtgcaggtctttacagattcttcggggattcttcaatgcagatagacccgtcaatgccgtcgcataacgtacctgtaccaccgacacccacacctcctgatacgcaaattgtatatggagatttgaacggcgaccagaaagtgacttccacagactatacgatgctcaagaggtatttgatgaaaagcattgataggtttaatacttccgaacaagctgcggatttgaacagagacggcaaaatcaattccacggacttgacaatattgaaaagatatttgctttacagcataccgtctctccctatataa

SEQ ID NO: 14 - protein, 602 aa, Clostridium thermocellum
  CelT-d1
MRGSHHHHHHTDPGIVSFNTVSTSAAGEYNYAKALQYSMFFYDANMCGTGVDENSLLSWRGDCHVYDARLPLDSQNTNMSDGFISSNRSVLDPDGDGKVDVSGGFHDAGDHVKFGLPEAYAASTVGWGYYEFKDQFRATGQAVHAEVILRYFNDYFMRCTFRDASGNVVAFCHQVGDGDIDHAFWGAPENDTMFRRGWFITKEKPGTDIISATAASLAINYMNFKDTDPQYAAKSLDYAKALFDFAEKNPKGVVQGEDGPKGYYGSSKWQDDYCWAAAWLYLATQNEHYLDEAFKYYDYYAPPGWIHCWNDVWSGTACILAEINDLYDKDSQNFEDRYKRASNKNQWEQIDFWKPIQDLLDKWSGGGITVTPGGYVFLNQWGSARYNTAAQLIALVYDKHHGDTPSKYANWARSQMDYLLGKNPLNRCYVVGYSSNSVKYPHHRAASGLKDANDSSPHKYVLYGALVGGPDASDQHVDRTNDYIYNEVAIDYNAAFVGACAGLYRFFGDSSMQIDPSMPSHNVPVPPTPTPPDTQIVYGDLNGDQKVTSTDYTMLKRYLMKSIDRFNTSEQAADLNRDGKINSTDLTILKRYLLYSIPSLPI

SEQ ID NO: 15 - DNA, 2400 bp, Clostridium thermocellum
  CelE-d1
atgagaggatcgcatcaccatcaccatcacggatccccggtaaaaggctttcaggtatcgggaacaaagcttttggatgcaagcggaaacgagcttgtaatgaggggcatgcgtgatatttcagcaatagatttggttaaagaaataaaaatcggatggaatttgggaaatactttggatgctcctacagagactgcctggggaaatccaaggacaaccaaggcaatgatagaaaaggtaagggaaatgggctttaatgccgtcagagtgcctgttacctgggatacgcacatcggacctgctccggactataaaattgacgaagcatggctgaacagagttgaggaagtggtaaactatgttcttgactgcggtatgtacgcgatcataaatcttcaccatgacaatacatggattatacctacatatgccaatgagcaaaggagtaaagaaaaacttgtaaaagtttgggaacaaatagcaacccgttttaaagattatgacgaccatttgttgtttgagacaatgaacgaaccgagagaagtaggttcacctatggaatggatgggcggaacgtatgaaaaccgagatgtgataaacagatttaatttggcggttgttaataccatcagagcaagcggcggaaataacgataaaagattcatactggttccgaccaatgcggcaaccggcctggatgttgcattaaacgaccttgtcattccgaacaatgacagcagagtcatagtatccatacatgcttattcaccgtatttctttgctatggatgtcaacggaacttcatattggggaagtgactatgacaaggcttctcttacaagtgaacttgatgctatttacaacagatttgtgaaaaacggaagggctgtaattatcggagaattcggaaccattgacaagaacaacctgtcttcaagggtggctcatgccgagcactatgcaagagaagcagtttcaagaggaattgctgttttctggtgggataacggctattacaatccgggtgatgcagagacttatgcattgctgaacagaaaaactctctcatggtattatcctgaaattgtccaggctcttatgagaggtgccggcgttgaacctttagtttcaccgactcctacacctacattaatgccgaccccctcgcccacggtgacagcaaatattttgtacggtgacgtaaacggggacggaaaaataaattctacagactgtacaatgctaaagagatatattttgcgtggcatagaagaattcccaagtcctagcggaattatagccgctgacgtaaatgcggatctgaaaatcaattccaccgacttggtattgatgaaaaaatatctactgcgctcaatagacaaatttcctgcggaggattctcaaacacctgatgaagacaatccgggcattttgtataacggaagattcgatttttcagatccgaacggtccgaaatgcgcctggtccggcagcaatgttgagctgaatttttacggcacggaagcaagtgtgactatcaaatccggcggtgagaactggttccaggctattgtagacggcaatcctcttcctcctttttcggttaacgctactacctctaccgtaaagcttgtaagcggtcttgcagaaggagctcatcatcttgtattgtggaagaggacagaggcatccttgggagaagttcagttccttgggtttgattttggttcaggaaagcttcttgccgcaccgaagcctttggaaagaaagattgagtttatcggagactccatcacatgtgcatacggaaatgaaggaacaagcaaggagcagtcttttacaccgaaaaatgaaaacagctatatgtcttatgcggcaattacagcccgtaatttgaatgcaagtgcaaatatgattgcgtggtccggaatcggacttaccatgaactacggcggagcccccggacctcttataatggaccgttatccttatacccttccttacagcggagtcagatgggattttagcaaatatgtgcctcaggttgttgtaatcaatcttggtaccaatgatttttctacatcatttgcagataaaacaaagtttgtaacggcatataaaaaccttataagtgaagttcgcaggaactatccggatgcccatatattctgctgtgtcggtccgatgctttggggaacgggcctggatttgtgccgcagttatgttacggaagttgtaaatgattgtaacagaagcggggatttaaaggtgtattttgttgagtttccgcagcaggacggaagcaccggatacggagaagactggcatccaagtattgccacccaccagctgatggctgagcggcttactgcggaaataaaaaacaagcttggatggtaa

SEQ ID NO: 16 - protein, 799 aa, Clostridium thermocellum
  CelE-d1
MRGSHHHHHHGSPVKGFQVSGTKLLDASGNELVMRGMRDISAIDLVKEIKIGWNLGNTLDAPTETAWGNPRTTKAMIEKVREMGFNAVRVPVTWDTHIGPAPDYKIDEAWLNRVEEVVNYVLDCGMYAIINLHHDNTWIIPTYANEQRSKEKLVKVWEQIATRFKDYDDHLLFETMNEPREVGSPMEWMGGTYENRDVINRFNLAVVNTIRASGGNNDKRFILVPTNAATGLDVALNDLVIPNNDSRVIVSIHAYSPYFFAMDVNGTSYWGSDYDKASLTSELDAIYNRFVKNGRAVIIGEFGTIDKNNLSSRVAHAEHYAREAVSRGIAVFWWDNGYYNPGDAETYALLNRKTLSWYYPEIVQALMRGAGVEPLVSPTPTPTLMPTPSPTVTANILYGDVNGDGKINSTDCTMLKRYILRGIEEFPSPSGIIAADVNADLKINSTDLVLMKKYLLRSIDKFPAEDSQTPDEDNPGILYNGRFDFSDPNGPKCAWSGSNVELNFYGTEASVTIKSGGENWFQAIVDGNPLPPFSVNATTSTVKLVSGLAEGAHHLVLWKRTEASLGEVQFLGFDFGSGKLLAAPKPLERKIEFIGDSITCAYGNEGTSKEQSFTPKNENSYMSYAAITARNLNASANMIAWSGIGLTMNYGGAPGPLIMDRYPYTLPYSGVRWDFSKYVPQVVVINLGTNDFSTSFADKTKFVTAYKNLISEVRRNYPDAHIFCCVGPMLWGTGLDLCRSYVTEVVNDCNRSGDLKVYFVEFPQQDGSTGYGEDWHPSIATHQLMAERLTAEIKNKLGW

SEQ ID NO: 17 - DNA, 2985 bp, Clostridium thermocellum
  CelQ-d1
atgagaggatctcaccatcaccatcaccatgggatccgcatgcgagctcggtaccccgggtcgacggcatttattcttcctcaggggattgtgtccgcagcaggaagctataactatgcggaagcacttcagaaagccatttacttttatgagtgtcagcaggccggccctctacctgaatggaaccgcgttgagtggcgtggcgacgcaacaatgaatgatgaggtacttggtggatggtatgacgcaggtgaccatgtcaagtttaatctgcctatggcgtattcggcggcaatgcttggctgggctctttatgagtatggcgatgacattgaggcatcggggcagagacttcatcttgaaaggaaccttgcctttgcccttgactatcttgttgcctgcgacagaggtgacagtgtcgtttatcagataggtgacggtgccgctgaccataaatggtggggttctgcggaagttattgaaaaagaaatgacaagaccttactttgtaggaaagggatccgccgttgtaggtcagatggctgcagctttggctgtaggttccatagttcttaaaaatgatacatacctcagatatgcgaagaagtatttcgaacttgcagatgcaacaagaagtgacagcacttatactgctgcaaatggtttctacagttcccacagcggattctgggatgagctgttgtgggcttccacttggctctatcttgcaacaggtgatagaaattatcttgataaagctgagtcctatattccaaaattaaaccgtcagaatcagaccacagatatagaatatcagtgggcacattgctgggatgactgccactatggagcaatgatcttgcttgcaagagctacaggtaaagaagagtatcacaaatttgcacaaatgcatctggattggtggacacctcaaggttataacggaaagagagttgcatatactcccggcggacttgcgcatcttgatacctggggaccgttgagatatgctacaactgaagcattcctcgcttttgtatatgccgattcaataaatgacccggctctcaagcaaaaatattataattttgcgaaaagccagattgactatgcattgggttcaaatcctgacaacagaagctatgtagtcggatttggaaacaatccgccacagcgtcctcaccacagaaccgctcatggaacttggttggataaaagagatattccggaaaagcacagacatgtactttacggtgctctggtcggaggacccggaagagatgacagttatgaagacaatatagaggattatgtaaaaaatgaagttgcctgcgactacaatgcaggttttgtaggcgcgctctgcagattgactgctgaatacggcggaactcctcttgcgaacttcccgccaccggaacaaagagatgatgagttcttcgtagaagcggctataaatcaggcaagtgatcatttcactgaaataaaagcattgctcaacaaccgttcatcctggccggcaagacttattaaggacctttcatacaactattatatggatttgactgaagtttttgaggcaggttacagtgttgacgatattaaagtaacaataggctattgcgaaagcggtatggatgtcgagatttcgccgattactcatttgtatgacaatatttattacataaaaatatcatatatcgacggaaccaatatttgtccgataggtcaggaacagtatgccgctgagcttcagttccgtattgcggcacctcaaggtactaaattctgggatccgacaaatgacttctcatatcagggacttaccagagagttggcaaagacaaaatatatgcccgtttttgacggagcaacaaaaatctttggagaagttccaggcggctttgaaccggttccttcaccttcgccgactcctgctcaatataaagtcggtgacttaaacggtgacggagtggttaattcaactgacagtgtaatattgaaaagacatataattaaattttctgaaataacagatccagttaaattgaaagctgctgatcttaacggagatggcaatataaactccagcgatgtttcattaatgaagagatatctgctccgtataatagataaatttccggtagaatagtttggcatttgaaataagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaa

SEQ ID NO: 18 - protein, 987 aa, Clostridium thermocellum
  CelQ-d1
MRGSHHHHHHGIRMRARYPGSTAFILPQGIVSAAGSYNYAEALQKAIYFYECQQAGPLPEWNRVEWRGDATMNDEVLGGWYDAGDHVKFNLPMAYSAAMLGWALYEYGDDIEASGQRLHLERNLAFALDYLVACDRGDSVVYQIGDGAADHKWWGSAEVIEKEMTRPYFVGKGSAVVGQMAAALAVGSIVLKNDTYLRYAKKYFELADATRSDSTYTAANGFYSSHSGFWDELLWASTWLYLATGDRNYLDKAESYIPKLNRQNQTTDIEYQWAHCWDDCHYGAMILLARATGKEEYHKFAQMHLDWWTPQGYNGKRVAYTPGGLAHLDTWGPLRYATTEAFLAFVYADSINDPALKQKYYNFAKSQIDYALGSNPDNRSYVVGFGNNPPQRPHHRTAHGTWLDKRDIPEKHRHVLYGALVGGPGRDDSYEDNIEDYVKNEVACDYNAGFVGALCRLTAEYGGTPLANFPPPEQRDDEFFVEAAINQASDHFTEIKALLNNRSSWPARLIKDLSYNYYMDLTEVFEAGYSVDDIKVTIGYCESGMDVEISPITHLYDNIYYIKISYIDGTNICPIGQEQYAAELQFRIAAPQGTKFWDPTNDFSYQGLTRELAKTKYMPVFDGATKIFGEVPGGFEPVPSPSPTPAQYKVGDLNGDGVVNSTDSVILKRHIIKFSEITDPVKLKAADLNGDGNINSSDVSLMKRYLLRIIDKFPVEFGINKLNLSLDSCIQPQNSIWICSERSVAAGRFLLVRIQASLARFSGAKEAKMEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 19 - DNA, 3696 bp, Clostridium thermocellum
  CbhA-d1
atgagaggatctcaccatcaccatcaccatacggatccgcatgcgagctccgtgtttgccttagaagataattcttcgactttgccgccgtataaaaacgaccttttgtatgagaggacttttgatgagggactttgttatccatggcatacctgtgaagacagcggaggaaaatgctcctttgatgtggtcgatgttccggggcagcccggtaataaagcatttgccgttactgttcttgacaaagggcaaaacagatggagagttcagatgagacaccgtggtcttactcttgaacagggacatacatatagagtacggcttaagatttgggcagatgcgtcctgtaaagtttatataaaaataggacaaatggcggagccctatgctgaatattggaacaacaagtggagtccatacacactgacagcaggtaaggtattggaaattgacgagacgtttgttatggacaagccaactgacgacacatgcgaatttacattccatttaggtggcgaattggcagcaactcctccatatacagtttatcttgatgatgtatccctttatgacccagaatatacgaagcctgttgaatatatacttccgcagcctgatgtacgtgtgaaccaggttggctacctgccggagggcaagaaagttgccactgtggtatgcaattcaactcagccggtaaaatggcagcttaagaatgctgcaggcgttgtagttttggaaggttataccgaaccaaagggtcttgacaaagactcgcaggattatgtacattggcttgatttttccgattttgcaaccgaaggaattggttactattttgaacttccgactgtaaacagtcctacaaactacagtcatccatttgacattcgcaaagacatctatactcagatgaaatatgatgcattggcattcttctatcacaagagaagcggtattcctattgaaatgccgtatgcaggaggagaacagtggaccagacctgcaggacatatcggaattgagccgaacaagggagatacaaatgttcctacatggcctcaggatgatgagtatgcaggaatacctcagaagaattatacaaaggatgtaaccggtggatggtatgatgccggtgaccacggtaaatatgttgtaaacggcggtatagccgtctggacattaatgaacatgtatgagagggcaaaaattagaggtcttgacaactggggaccatacagggacggcggaatgaacataccggagcagaataacggttatccggacattcttgatgaagcaagatgggaaattgagttctttaagaaaatgcaggtaactgaaaaagaggatccttccatagccggaatggtacaccacaaaattcacgacttcagatggactgctttgggtatgttgcctcacgaagatccccagccacgttacttaaggccggtaagtacggctgcgactttgaactttgcggcaactttggcacaaagtgcacgtctttggaaagattatgatccgacttttgctgctgactgtttggaaaaggctgaaatagcatggcaggcggcattaaagcatcctgatatttatgctgagtatactcccggtagcggtggtcccggaggcggaccatacaatgacgactatgtcggagacgaattctactgggcagcctgcgaactttatgtaacaacaggaaaagacgaatataagaattacctgatgaattcacctcactatcttgaaatgcctgcaaagatgggtgaaaacggtggagcaaacggagaagacaacggattgtggggatgcttcacctggggaactactcaaggattgggaaccattactcttgctttggttgaaaacggattgcctgctacagacattcaaaaggcaagaaacaatatagctaaagctgctgacagatggcttgagaatattgaagagcaaggttacagactgccgatcaaacgggcggaggatgagagagccggttatccatggggttcaaactccttgcattttgaaccagatgacctagttatgggatatgcctatgactttacaggtgactcaaatatctcgatggaatgtttgaccggcataagctacctgttgggaagaaacgcaatggatcagtcctatgtaacagggtatggtgagcgtccgcttcagaatcctcatgacaggttctggacgccgcagacaagtaagagattccctgctccacctccgggtataatttccggccgtccgaactcccgtttcgaggacccgacaataaatgcggccgttaagaaggatacaccgccacagaaatgttttatcgaccatacagactcatggtcaaccaacgagataactgttaactggaatgctccgtttgcatgggttacagcttatcttgacgagcagtacacagacagtgaaaccgataaggtaactattgattcgcctgttgcaggagaaagatttgaagcgggtaaagacattaatataagaactgttaaatcaaaaactcctgtaagcaaagtagagttttacaatggagatacgcttatttccagtgacacaactgcaccttacacagcaaagataacaggagccgctgtcggagcatataaccttaaagcggttgcagtgctgtctgacggaagaagaattgagtcaccggtaactcctgtacttgttaaggtaattgtgaaacctactgtaaaacttactgcacccaagtcaaatgttgtggcttatggaaatgagttcctgaagattacagcaacagccagtgactctgacggcaaaatctccagggttgatttccttgttgacggtgaagtaatcggttcagacagggaagcaccttatgaatatgagtggaaagctgtggaaggcaatcacgaaataagtgtaattgcttatgatgatgacgatgcggcttcaacacctgattccgtaaaaatatttgtaaaacaggcacgggatgtaaaagtacagtatttgtgcgaaaatacgcaaacatccactcaggaaatcaagggtaaattcaatatagttaacacaggaaacagagattattcgctgaaagatatagtattaagatactactttaccaaggagcacaattcacagcttcagtttatctgctattatacacccataggctccggaaatctcattccgtcctttggcggctcgggtgacgagcattatctgcagctggaattcaaagatgtcaagctgcctgccggcggtcagactggggaaatacagtttgttataagatatgcagataactccttccatgatcagtcgaacgactattcgttcgatccaactataaaagcgttccaggattatggcaaggttaccctgtataagaatggagaacttgtttggggaacgccgccgggcggtacagaacctgaagaaccggaagagcctgaagaaccggaagagcctgcgatagtttacggcgactgtaatgatgacggcaaagtaaattcaacagacgtcgcagtaatgaagagatatttaaagaaagaaaatgttaatattaatcttgacaatgcagatgtgaatgcggacggcaaagttaactcaacagacttctcaatacttaagagatatgttatgaagaacatagaagaattgccatatcgataagataatctgaaattatttgtgtaa

SEQ ID NO: 20 - protein, 1223 aa, Clostridium thermocellum
  CbhA-d1
MRGSHHHHHHTDPHASSVFALEDNSSTLPPYKNDLLYERTFDEGLCYPWHTCEDSGGKCSFDVVDVPGQPGNKAFAVTVLDKGQNRWRVQMRHRGLTLEQGHTYRVRLKIWADASCKVYIKIGQMAEPYAEYWNNKWSPYTLTAGKVLEIDETFVMDKPTDDTCEFTFHLGGELAATPPYTVYLDDVSLYDPEYTKPVEYILPQPDVRVNQVGYLPEGKKVATVVCNSTQPVKWQLKNAAGVVVLEGYTEPKGLDKDSQDYVHWLDFSDFATEGIGYYFELPTVNSPTNYSHPFDIRKDIYTQMKYDALAFFYHKRSGIPIEMPYAGGEQWTRPAGHIGIEPNKGDTNVPTWPQDDEYAGIPQKNYTKDVTGGWYDAGDHGKYVVNGGIAVWTLMNMYERAKIRGLDNWGPYRDGGMNIPEQNNGYPDILDEARWEIEFFKKMQVTEKEDPSIAGMVHHKIHDFRWTALGMLPHEDPQPRYLRPVSTAATLNFAATLAQSARLWKDYDPTFAADCLEKAEIAWQAALKHPDIYAEYTPGSGGPGGGPYNDDYVGDEFYWAACELYVTTGKDEYKNYLMNSPHYLEMPAKMGENGGANGEDNGLWGCFTWGTTQGLGTITLALVENGLPATDIQKARNNIAKAADRWLENIEEQGYRLPIKRAEDERAGYPWGSNSLHFEPDDLVMGYAYDFTGDSNISMECLTGISYLLGRNAMDQSYVTGYGERPLQNPHDRFWTPQTSKRFPAPPPGIISGRPNSRFEDPTINAAVKKDTPPQKCFIDHTDSWSTNEITVNWNAPFAWVTAYLDEQYTDSETDKVTIDSPVAGERFEAGKDINIRTVKSKTPVSKVEFYNGDTLISSDTTAPYTAKITGAAVGAYNLKAVAVLSDGRRIESPVTPVLVKVIVKPTVKLTAPKSNVVAYGNEFLKITATASDSDGKISRVDFLVDGEVIGSDREAPYEYEWKAVEGNHEISVIAYDDDDAASTPDSVKIFVKQARDVKVQYLCENTQTSTQEIKGKFNIVNTGNRDYSLKDIVLRYYFTKEHNSQLQFICYYTPIGSGNLIPSFGGSGDEHYLQLEFKDVKLPAGGQTGEIQFVIRYADNSFHDQSNDYSFDPTIKAFQDYGKVTLYKNGELVWGTPPGGTEPEEPEEPEEPEEPAIVYGDCNDDGKVNSTDVAVMKRYLKKENVNINLDNADVNADGKVNSTDFSILKRYVMKNIEELPYR

SEQ ID NO: 21 - DNA, 2106 bp, Clostridium thermocellum
  c3-c1-c1-d2
atgagaggatctcaccatcaccatcaccatacggatccgccggcgggtattgcacgcgcagataaagcctcgagcattgagcttaagtttgaccgcaataagggcgaagttggagatatacttattggtaccgtacgcattaacaatatcaagaatttcgcaggctttcaggtaaacattgtatatgatccaaaagtcttaatggctgttgaccctgaaacggggaaagaatttacttcttcaacatttccgccaggccgcactgtactgaaaaacaatgcttacggcccaattcagattgcggacaatgatccggaaaaagggattctgaacttcgcgcttgcatattcatatattgcgggctacaaagaaacaggcgtagcggaggaaagcggcatcattgcgaaaattggctttaaaattctccagaaaaagagcactgccgtaaaattccaggatacattaagcatgcccggcgctatttcgggcacacagctgtttgactgggacggcgaagttattaccggctatgaggtaattcagccggatgtgctgagtttgggtgacgagccttatgagacaccgggcacggatattccgatttccgacaatccggcagcaactccgtcatccacgccgtcagttactccttcaccggatccgcccaccaggccatcggtaccgacaaacacaccgacaaacacaccggcaaatacaccggtatcaggcaatttgaaggttgaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatccgaatgcaataaagattaaggtggacacagtaaatgcaaaaccgggagacacagtaaatatacctgtaagattcagtggtataccatccaagggaatagcaaactgtgactttgtatacagctatgacccgaatgtacttgagataatagagataaaaccgggagaattgatagttgacccgaatcctgacaagagctttgatactgcagtatatcctgacagaaagataatagtattcctgtttgcagaagacagcggaacaggagcgtatgcaataactaaagacggagtatttgctacgatagtagcgaaagtaaaatccggagcacctaacggactcagtgtaatcaaatttgtagaagtaggcggatttgcgaacaatgaccttgtagaacagaggacacagttctttgacggtggagtaaatgttggagatacaacagtacctacaacacctacaacacctgtaacaacaccgacagattgttcgagctcagccaatgtaccgtcacatggtgtagtggtattaaaagtacaagcaagctccactgatacaaatattgagtttggtgatgttgacggcaatggcatgattgacgcattagattattcattagtaaaacggtatttgctgggccagatttctgattgtcctgattcaaaaggcaagcttgctgctgatgttgatggcgaccagcaaattacagcactggatttttcattaattaagcaatacttacttgggactattaacaaatttcctgctcaaacagcaagtcgacctgcagccaagcttaattag

SEQ ID NO: 22 - protein, 701 aa, Clostridium thermocellum
  c3-c1-c1-d2
MRGSHHHHHHTDPPAGIARADKASSIELKFDRNKGEVGDILIGTVRINNIKNFAGFQVNIVYDPKVLMAVDPETGKEFTSSTFPPGRTVLKNNAYGPIQIADNDPEKGILNFALAYSYIAGYKETGVAEESGIIAKIGFKILQKKSTAVKFQDTLSMPGAISGTQLFDWDGEVITGYEVIQPDVLSLGDEPYETPGTDIPISDNPAATPSSTPSVTPSPDPPTRPSVPTNTPTNTPANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDDPNAIKIKVDTVNAKPGDTVNIPVRFSGIPSKGIANCDFVYSYDPNVLEIIEIKPGELIVDPNPDKSFDTAVYPDRKIIVFLFAEDSGTGAYAITKDGVFATIVAKVKSGAPNGLSVIKFVEVGGFANNDLVEQRTQFFDGGVNVGDTTVPTTPTTPVTTPTDCSSSANVPSHGVVVLKVQASSTDTNIEFGDVDGNGMIDALDYSLVKRYLLGQISDCPDSKGKLAADVDGDQQITALDFSLIKQYLLGTINKFPAQTASRPAAKLN

SEQ ID NO: 23 - DNA, 1893 bp, Clostridium thermocellum
  CBM-c1-c1-d3
atgagaggatctcaccatcaccatcaccatacggatccggtatcaggcaatttgaaggttgaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatccgaatgcaataaagattaaggtggacacagtaaatgcaaaaccgggagacacagtaaatatacctgtaagattcagtggtataccatccaagggaatagcaaactgtgactttgtatacagctatgacccgaatgtacttgagataatagagataaaaccgggagaattgatagttgacccgaatcctgacaagagctttgatactgcagtatatcctgacagaaagataatagtattcctgtttgcagaagacagcggaacaggagcgtatgcaataactaaagacggagtatttgctacgatagtagcgaaagtaaaatccggagcacctaacggactcagtgtaatcaaatttgtagaagtaggcggatttgcgaacaatgaccttgtagaacagaggacacagttctttgacggtggagtaaatgttggagatacaacagtacctacaacacctacaacacctgtaacaacaccgacagatgattcgaatgcagtaaggattaaggtggacacagtaaatgcaaaaccgggagacacagtaagaatacctgtaagattcagcggtataccatccaagggaatagcaaactgtgactttgtatacagctatgacccgaatgtacttgagataatagagatagaaccgggagacataatagttgacccgaatcctgacaagagctttgatactgcagtatatcctgacagaaagataatagtattcctgtttgcggaagacagcggaacaggagcgtatgcaataactaaagacggagtatttgctacgatagtagcgaaagtaaaatccggagcacctaacggactcagtgtaatcaaatttgtagaagtaggcggatttgcgaacaatgaccttgtagaacagaagacacagttctttgacggtggagtaaatgttggagatacaacagaacctgcaacacctacaacacctgtaacaacaccgacaacaacagatgagctcgtaattgcaaatgttgtagtaacgggcgatacttcagtttcaacttcacaggctccaattatgatgtgggtaggcgacattgtgaaagacaattctatcaacctgttggacgttgcagaagttatccgttgcttcaacgctactaaaggcagcgcaaactacgtagaagaacttgacattaatcgcaacggcgcaattaacatgcaagacattatgattgttcataagcactttggcgctacatcaagtgattacgtcgacctgcagccaagcttaattagctga

SEQ ID NO: 24 - protein, 630 aa, Clostridium thermocellum
  CBM-c1-c1-d3
MRGSHHHHHHTDPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDDPNAIKIKVDTVNAKPGDTVNIPVRFSGIPSKGIANCDFVYSYDPNVLEIIEIKPGELIVDPNPDKSFDTAVYPDRKIIVFLFAEDSGTGAYAITKDGVFATIVAKVKSGAPNGLSVIKFVEVGGFANNDLVEQRTQFFDGGVNVGDTTVPTTPTTPVTTPTDDSNAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKSGAPNGLSVIKFVEVGGFANNDLVEQKTQFFDGGVNVGDTTEPATPTTPVTTPTTTDELVIANVVVTGDTSVSTSQAPIMMWVGDIVKDNSINLLDVAEVIRCFNATKGSANYVEELDINRNGAINMQDIMIVHKHFGATSSDYVDLQPSLIS

SEQ ID NO: 25 - DNA, 1767 bp, Clostridium thermocellum
  c2-c1-c1
atgagaggatctcaccatcaccatcaccatacggatcctaaattgactatcaatgtaggtgaaagtggtaatacaaatggtcttaaggtttcagtaggcacagctgttggtgctcctggtgatacagtaacagttcctgttacatttgctgatgtagcaaaagtaaacaacgtaggaacatgtaacttctatcttggctatgatgcaagtcttttggatgtagtatcagtagatgcaggtccaattgttaagaatgcagcagtaaacttctcaagcagtgcaagcaacggcacaatcagcttcctgttcttggacaacacaatcactgatgaattgattacttcagatggtgtgttcgcaaatatcacatttaagattaagagtactgctacacaaggtacaacaccaattaccttcaaagatggcggtgcttttggtgacggtactatgtcaaagattgcttcagttattaagacaagtggtagtgtagttattagtccagatcctacaaatgctcttaaagtaacagtaggcacagcagaaggtaatgttggcgaaacagtaacagttcctgttacatttgcggatccgcccaccaggccatcggtaccgacaaacacaccgacaaacacaccggcaaatacaccggtatcaggcaatttgaaggttgaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatccgaatgcaataaagattaaggtggacacagtaaatgcaaaaccgggagacacagtaaatatacctgtaagattcagtggtataccatccaagggaatagcaaactgtgactttgtatacagctatgacccgaatgtacttgagataatagagataaaaccgggagaattgatagttgacccgaatcctgacaagagctttgatactgcagtatatcctgacagaaagataatagtattcctgtttgcagaagacagcggaacaggagcgtatgcaataactaaagacggagtatttgctacgatagtagcgaaagtaaaatccggagcacctaacggactcagtgtaatcaaatttgtagaagtaggcggatttgcgaacaatgaccttgtagaacagaggacacagttctttgacggtggagtaaatgttggagatacaacagtacctacaacacctacaacacctgtaacaacaccgacagattgttcgagctcggtaccccgggtcgacctgcagccaagcttaattagctga

SEQ ID NO: 26 - protein, 588 aa, Clostridium thermocellum
  c2-c1-c1
MRGSHHHHHHTDPKLTINVGESGNTNGLKVSVGTAVGAPGDTVTVPVTFADVAKVNNVGTCNFYLGYDASLLDVVSVDAGPIVKNAAVNFSSSASNGTISFLFLDNTITDELITSDGVFANITFKIKSTATQGTTPITFKDGGAFGDGTMSKIASVIKTSGSVVISPDPTNALKVTVGTAEGNVGETVTVPVTFADPPTRPSVPTNTPTNTPANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDDPNAIKIKVDTVNAKPGDTVNIPVRFSGIPSKGIANCDFVYSYDPNVLEIIEIKPGELIVDPNPDKSFDTAVYPDRKIIVFLFAEDSGTGAYAITKDGVFATIVAKVKSGAPNGLSVIKFVEVGGFANNDLVEQRTQFFDGGVNVGDTTVPTTPTTPVTTPTDCSSSVPRVDLQPSLIS